(19) United States
(12) Patent Application Publication
(10) Pub. No.: US 2022/0354847 A1
(43) Pub. Date: Nov. 10, 2022

(54) THIAMINE THERAPY FOR FATTY LIVER ASSOCIATED DISEASES

(71) Applicant: THE STATE OF ISRAEL, MINISTRY OF AGRICULTURE & RURAL DEVELOPMENT, AGRICULTURAL RESEARCH ORGANIZATION, Rishon LeZion (IL)

(72) Inventors: Hay DVIR, Haifa (IL); Samuel BOCOBZA, Tel Aviv (IL)

(73) Assignee: THE STATE OF ISRAEL, MINISTRY OF AGRICULTURE & RURAL DEVELOPMENT, AGRICULTURAL RESEARCH ORGANIZATION, Rishon Lezion (IL)

(21) Appl. No.: 17/762,747

(22) PCT Filed: Sep. 24, 2020
(86) PCT No.: PCT/IL2020/051043

§ 371 Date: Mar. 23, 2022

Related U.S. Application Data
(60) Provisional application No. 62/906,111, filed on Sep. 26, 2019.

(51) Int. Cl. A61K 31/51; A61P 3/00; A61P 1/16; A61K 45/06

(52) U.S. Cl. CPC A61K 31/51; A61P 3/00; A61P 1/16; A61K 45/06

(57) ABSTRACT

The disclosure relates to compositions comprising thiamine, a thiamine ester, or a thiamine analog, and to methods for preventing and treating fatty-liver diseases.

### BACKGROUND OF THE DISCLOSURE

Fatty Liver (FL), also known as hepatic steatosis, develops when the rate of hepatic synthesis and uptake of fatty acids (FA) exceeds the rate of their breakdown and export out of the liver. The resulting excess amount of hepatic FA is directed toward triglycerides (TG) synthesis that accumulate in cytoplasmic lipid droplets of liver cells.

FL has long been linked to excess alcohol consumption. However, the prevalence of non-alcoholic FL disease (NAFLD) has increased dramatically in the past few decades, in parallel to the rise in obesity, metabolic syndrome (MetS) and type-2 diabetes mellitus (T2D). In fact, incidents of NAFLD had reached epidemic levels estimated at ˜25% globally. A few gene-specific polymorphisms have been associated with NAFLD, yet it is clearly a multifactorial disorder with a substantial environmental component, stimulated primarily by excessive caloric intake and a sedentary lifestyle.

Somewhat less intuitive, FL and liver-related metabolic pathologies can also develop due to malnutrition in various domestic animals including ruminants, cats and birds. In particular, the physiology of negative energy balance is well recognized in transitioning ruminants, overweight cats undergoing periods of anorexia with little or no food intake, as well as in pregnant women undergoing sudden diarrhea and vomiting episodes (clinically known as pregnancy starvation ketoacidosis). In such conditions, extensive adipose lipolysis takes place to meet with the physiological energy demands. The fat released from adipose tissue is mobilized into circulation, in the form of non-esterified fatty acids (NEFA), for uptake and catabolism in the liver. Yet, extensive NEFA uptake by the liver can exceeds its capacity to catabolize them, shifting them toward triglyceride synthesis and accumulation as fat in hepatocytes lipid droplets. In addition, both alcoholic liver disease (ALD) and NAFLD are tightly linked to MetS and T2D; collectively suggesting that hepatic steatosis can lead to liver damage and metabolic dysfunction irrespective of its origin.

By histopathology, NAFLD is defined as the presence of ≥5% steatotic hepatocytes in the absence of secondary causes such as significant alcohol consumption or the use of steatogenic medications. The less subjective biochemical threshold refers to having a total TG weight greater than 5.5% of the wet liver weight. Yet, NAFLD refers not only to the condition of hepatic steatosis but also to the more progressive pathogenic states, i.e. non-alcoholic steatohepatitis (NASH) and cirrhosis, involving inflammation and tissue-destructive fibrosis that enhance the odds of developing hepatocellular carcinoma (HCC). The strong association of NAFLD with systemic metabolic dysregulation, such as hypertension, dyslipidemia, hyperglycemia, and most predominantly with insulin resistance, enhanced the notion that NAFLD is a multisystem disorder affecting extrahepatic organs. As such, it has been considered as a precursor for MetS or as the liver-associated manifestation of the MetS. Consistently, cardiovascular disease is the leading cause of mortality among NAFLD patients, followed by liver- and cancer-related mortality.

Despite of the major health concern with the increasing worldwide prevalence of NAFLD and the accompanying rise in end-stage liver disease requiring liver transplantation and in HCC as a result of NAFLD even in the absence of cirrhosis, there is yet no approved drug therapy. Recommended management of NAFLD consists of treating both the liver disease as well as the associated metabolic comorbidities. Weight loss achieved by calorie-restricted diet, alone or combined with exercise, has proven beneficial in improving hepatic steatosis and NASH scores as well as in reducing insulin resistance. Currently, this is the most recommended treatment, however, patient adherence to the required long-term lifestyle modifications is notoriously poor.

Whereas numerous therapeutic strategies are actively being developed and tested for targeting the steatotic, oxidative, inflammatory, fibrotic, and metabolic aspects of NAFLD, the response levels seen in clinical trials have been inadequate. This may reflect the heterogeneous and multifaceted nature of this disease and highlights the need for more robust preclinical models and for combination therapies. Likewise, therapeutic agents of potential to counteract multiple aspects of this complex disorder may be of high clinical value.

Pharmacological interventions based on insulin sensitizers, e.g. pioglitazone, and on antioxidants, e.g. vitamin E, have been shown to improve liver histology and are now recommended for biopsy-proven NASH patients. However, the long-term efficacy and safety of these treatments need to be assessed, as they have been associated with weight gain and a modest increase in the risk for prostate cancer, respectively. Additional strategies for targeting hepatic fat accumulation, de-novo lipogenesis, fibrosis, the intestinal microbiome and the metabolic comorbidities are under evaluation with the hope to identify potential drugs and/or combination therapies to combat the NAFLD epidemic. There is currently no FDA-approved drug for NAFLD.

Systemic metabolic morbidity, such as insulin resistance and hyperlipidemia, which are common to obesity, T2D and NAFLD, stimulated investigations of drugs approved for obesity and T2D for their benefit to NAFLD. However, the success rate was limited. For instance, metformin, a very common and efficient antidiabetic drug, was found to have no effect on hepatic steatosis (Biomedical Reports, 2013, 1: 57-64). Sitagliptin, another anti-diabetic drug, was found to be ineffective in the reduction of liver fat (Journal of Hepatology, 2016, 65, 369-376). Similarly, statins therapy, which is recommended to reduce the risk for cardiovascular disease in obese and MetS patients presenting dyslipidemia, showed variable adverse effects on liver histology and is avoided in NAFLD patients with advanced liver damage. Vitamin E which was suggested for the treatment of NAFLD in biopsy-proven NASH patients, was not suggested for people with diabetes (Hepatology, 2018, 67, 1, 328-357).

Thus, despite certain overlapping pathologies between metabolic disorders, one cannot trivially assume that drugs effective in one of them will be of benefit to another. Instead, the specific benefits of each potential drug candidate from one disorder need to be validated in the setting of another.

A myriad of NAFLD animal models (mostly in mice) have contributed significantly to the understanding of the disease etiology. Each model shows a better resemblance to certain aspects of this multifactorial disease. It should be noted, however, that preclinical results obtained in rodent models of NAFLD have repeatedly failed to be translated in human clinical trials. Therefore, the development of more robust models with better translational potential, as large animals often offer, is of significant biomedical and clinical value.

Large animal models convey a few additional advantages of physiological and experimental significance such as longer life span, similar body weight, and substantially larger quantities of biological material that allow for studying long-term effects and for frequent sampling of the same individual animal, respectively.

Interestingly, ruminants are susceptible to spontaneous development of FL during the transition period (late pregnancy and early lactation). This FL pathology is associated with decreased liver function, leading to severe and often lethal metabolic disorders, i.e. pregnancy toxemia and/or ketosis. Somewhat less intuitively, FL in transitioning ruminants develops primarily due to insufficient dietary energy consumption, leading to enhanced adipose lipolysis and fat mobilization in the circulation to the liver in the form of non-esterified fatty acids (NEFA). Although pregnancy- or starvation-related steatosis does not reflect the typical background for the development of NAFLD based on over nutrition, its spontaneous development in sheep, together with productive studies modeling obesity and diabetes in sheep, enhance the attractiveness of using sheep as a model for NAFLD.

There remains a need to identify new agents for treating FL and FL-related diseases. The use of large animal models in identifying these agents may beneficially increase their likelihood of remaining clinically relevant in humans.

### SUMMARY OF THE DISCLOSURE

The present inventors recently reported a large-animal nutritional model for FL, characterized by excessive carbohydrates intake, hyperglycemia, hyperinsulinemia and insulin resistance, which collectively drove liver steatosis in sheep (Kalyesubula et al, Sci Rep, 2020). In addition to the relevant MetS-like nature of this model, the use of large-animal models for preclinical testing of potential therapies increases their clinical relevance due to the similar body and organ weights and size.

In search for a pharmacological intervention to counteract both the systemic and liver-related metabolic aspects of this model, thiamine, also known as vitamin B1, was found as a plausible beneficial agent due to its fundamental role in energy metabolism. Thiamine, thiamine esters, and thiamine analogs, are active agents according to the principles of the present invention.

The present disclosure provides compositions and methods for preventing and treating fatty liver, fatty liver associated symptoms and fatty liver associated diseases in humans and animals.

The present invention provides, in one aspect, a method of preventing or treating a condition selected from the group consisting of: (i) fatty liver (FL) disease (FLD), (ii) a FL-associated disease, and (iii) a FL-associated symptom, in a patient in need of such prevention or treatment, the method comprising the step of administering a therapeutically effective amount of thiamine, a thiamine ester, or a thiamine analog, to the patient.

In certain embodiments, the condition is FLD.

In certain embodiments, the condition is a FL-associated disease selected from the group consisting of non-alcoholic fatty liver disease (NAFLD), alcoholic fatty liver disease, non-alcoholic steatohepatitis (NASH), metabolic syndrome (MetS), type-2 diabetes mellitus (T2D), cardiovascular disease, dyslipidemia, alcoholic liver disease, hepatocellular carcinoma (HCC), viral steatohepatitis, overnutrition, overweight, and obesity.

In certain embodiments, the condition is NAFLD. In certain embodiments, the condition is alcoholic fatty liver disease. In certain embodiments, the condition is NASH. In certain embodiments, the condition is MetS. In certain embodiments, the condition is T2D. In certain embodiments, the condition is cardiovascular disease. In certain embodiments, the condition is dyslipidemia. In certain embodiments, the condition is alcoholic liver disease. In certain embodiments, the condition is HCC. In certain embodiments, the condition is viral steatohepatitis. In certain embodiments, the condition is overnutrition. In certain embodiments, the condition is overweight. In certain embodiments, the condition is obesity.

In certain embodiments, the condition is a FL-associated symptom selected from the group consisting of hepatic steatosis, hepatic cirrhosis, hepatic inflammation, hepatic fibrosis, hepatic pain, hepatic yellow pigmentation, decreased liver function, low liver glycogen level, tiredness, systemic insulin resistance, hyperglycemia, systemic hypertriglyceridemia, micro-vesicular steatosis, macro-vesicular steatosis, high food intake, and high weight gain.

In certain embodiments, the condition is hepatic steatosis. In certain embodiments, the condition is hepatic cirrhosis. In certain embodiments, the condition is hepatic inflammation. In certain embodiments, the condition is hepatic fibrosis. In certain embodiments, the condition is hepatic pain. In certain embodiments, the condition is hepatic yellow pigmentation. In certain embodiments, the condition is decreased liver function. In certain embodiments, the condition is low liver glycogen level. In certain embodiments, the condition is tiredness. In certain embodiments, the condition is systemic insulin resistance. In certain embodiments, the condition is hyperglycemia. In certain embodiments, the condition is systemic hypertriglyceridemia. In certain embodiments, the condition is micro-vesicular steatosis. In certain embodiments, the condition is macro-vesicular steatosis. In certain embodiments, the condition is high food intake. In certain embodiments, the condition is high weight gain.

In certain embodiments, the hepatic steatosis is at least 5.5% by weight fat content (wet/wet).

In certain embodiments, the patient is afflicted with Type-2 diabetes mellitus (T2D) and/or insulin resistance. In certain embodiments, the patient is afflicted with T2D. In certain embodiments, the patient is afflicted with insulin resistance. In certain embodiments, the patient is afflicted with T2D and insulin resistance.

In certain embodiments, the patient is not afflicted with Type-2 diabetes mellitus (T2D) and/or insulin resistance.

In certain embodiments, the patient is not afflicted with T2D. In certain embodiments, the patient is not afflicted with insulin resistance. In certain embodiments, the patient is not afflicted with T2D and is not afflicted with insulin resistance.

In certain embodiments, the patient is suffering from FL and obesity and/or insulin resistance. In certain embodiments, the patient is suffering from FL and obesity. In certain embodiments, the patient is suffering from FL and insulin resistance. In certain embodiments, the patient is suffering from FL and obesity and insulin resistance. In certain embodiments, the patient is suffering from FL and T2D.

In certain embodiments, the method comprises administering about 200 mg thiamine, a thiamine ester, or a thiamine analog, to about 500 mg thiamine, a thiamine ester, or a thiamine analog, every day. In certain embodiments, the method comprises administering about 300 mg thiamine, a thiamine ester, or a thiamine analog, to about 400 mg thiamine, a thiamine ester, or a thiamine analog, every day.

In certain embodiments, the method comprises administering about 200 mg thiamine to about 500 mg thiamine, every day. In certain embodiments, the method comprises administering about 200 mg thiamine ester to about 500 mg thiamine ester, every day. In certain embodiments, the method comprises administering about 200 mg thiamine analog to about 500 mg thiamine analog, every day. In certain embodiments, the method comprises administering about 300 mg thiamine to about 400 mg thiamine, every day. In certain embodiments, the method comprises administering about 300 mg thiamine ester to about 400 mg thiamine ester, every day. In certain embodiments, the method comprises administering about 300 mg thiamine analog to about 400 mg thiamine analog, every day.

In certain embodiments, the thiamine, a thiamine ester, or a thiamine analog, is administered to the patient by oral administration, intravenous administration or intramuscular administration.

In certain embodiments, the thiamine, a thiamine ester, or a thiamine analog, is administered to the patient by oral administration. In certain embodiments, the thiamine, a thiamine ester, or a thiamine analog, is administered to the patient by intravenous administration. In certain embodiments, the thiamine, a thiamine ester, or a thiamine analog, is administered to the patient by intramuscular administration.

In certain embodiments, the method comprises administering thiamine.

In certain embodiments, the method comprises administering a thiamine ester.

In certain embodiments, the thiamine ester is selected from the group consisting of thiamine monophosphate and thiamine pyrophosphate.

In certain embodiments, the thiamine ester is thiamine monophosphate. In certain embodiments, the thiamine ester is thiamine pyrophosphate.

In certain embodiments, the method comprises administering a thiamine analog.

In certain embodiments, the thiamine analog is selected from the group consisting of Acefurtiamine, Acetiamine, Allithiamine, Beclotiamine, Benfotiamine, Bentiamine, Bisbentiamine, Cetotiamine, Cycotiamine, Fursultiamine, Monophosphothiamine, Octotiamine, Prosultiamine, Sulbutiamine, and Vintiamol.

In certain embodiments, the thiamine analog is Acefurtiamine. In certain embodiments, the thiamine analog is Acetiamine. In certain embodiments, the thiamine analog is Allithiamine. In certain embodiments, the thiamine analog is Beclotiamine. In certain embodiments, the thiamine analog is Benfotiamine. In certain embodiments, the thiamine analog is Bentiamine. In certain embodiments, the thiamine analog is Bisbentiamine. In certain embodiments, the thiamine analog is Cetotiamine. In certain embodiments, the thiamine analog is Cycotiamine. In certain embodiments, the thiamine analog is Fursultiamine. In certain embodiments, the thiamine analog is Monophosphothiamine. In certain embodiments, the thiamine analog is Octotiamine. In certain embodiments, the thiamine analog is Prosultiamine. In certain embodiments, the thiamine analog is Sulbutiamine. In certain embodiments, the thiamine analog is Vintiamol.

The present invention further provides, in another aspect, a composition comprising thiamine, a thiamine ester, or a thiamine analog, and at least one additional agent selected from the group consisting of an insulin sensitizer, an insulin-releasing enhancer, and an antioxidant.

In certain embodiments, the insulin sensitizer is selected from the group consisting of a Biguanide, a Thiazolidinedione, and a Lyn kinase activator.

In certain embodiments, the insulin sensitizer is a Biguanide. In certain embodiments, the insulin sensitizer is a Thiazolidinedione. In certain embodiments, the insulin sensitizer is a Lyn kinase activator.

In certain embodiments, the Biguanide is Metformin.

In certain embodiments, the Thiazolidinedione is Pioglitazone.

In certain embodiments, the Lyn kinase activator is Tolimidone.

In certain embodiments, the insulin-releasing enhancer is a glucagon-like peptide-1 receptor (GLP-1 receptor) agonist.

In certain embodiments, the GLP-1 receptor agonist is liraglutide.

In certain embodiments, the antioxidant is vitamin E.

The present invention further provides, in another aspect, a dosage form, comprising any one of the compositions described above.

The present invention further provides, in another aspect, a method for inducing fatty liver (FL), a FL-associated disease, or a FL-associated symptom, in a large animal, comprising the step of administering a high-calorie diet to the animal.

In certain embodiments, the animal is a sheep.

In certain embodiments, the high-calorie diet comprises an average daily metabolizable energy of 6.3 MCal.

In certain embodiments, the method further comprises administering a drug to the animal.

In certain embodiments, the method further comprises testing the effect of the drug on the fatty liver (FL), the FL-associated disease, or the FL-associated symptom, in the animal.

### BRIEF DESCRIPTION OF THE DRAWINGS

FIG. 1. Glucose response to the high-calorie and low-calorie dietary treatments. Repeated measures ANOVA detects an effect of treatment (P<0.0001), time (P<0.0001), treatment-time interaction (P<0.0001) and individual animal (P=0.001). SEM=0.638. The fasting data, at day 115 from treatment, were not included in the analysis.

FIG. 2. Plasma triglyceride response to the high-calorie and the low-calorie dietary treatments. Due to unequal variances between treatments, the statistical analysis was performed on the log-transformed values. Repeated measures ANOVA detects an effect of treatment (P=0.001), time (P<0.0001), and treatment by time interaction (P=0.01). SEM=0.716. Fasting data were not included in the model and were analyzed independently by a student's t-test, and the significance level was Bonferroni-Holm corrected. *P<0.05.

FIG. 3. Hepatic index (liver weight per body weight in percentage) in the high- and low-calorie treatments. A Student's t-test detects an effect of treatment (P<0.0001).

FIG. 4. Insulin response to the high-calorie and low-calorie treatments. Repeated measures ANOVA detects an effect of treatment (P<0.0001), time (P<0.0001), and individual animal (P=0.01) but no treatment by time interaction. SEM=8.29. Due to unequal variances between treatments, the statistical analysis was performed on the log-transformed values. Fasting insulin concentration was higher in the HC group (P=0.0002) but excluded for the ANOVA with repeated measures analysis.

FIG. 5. Non-esterified fatty acid (NEFA) response to the high-calorie and low-calorie treatments. Repeated measures ANOVA detected an effect of treatment (P<0.0001) and time (P<0.009). SEM=16.9. Fasting levels were not included in the model and analyzed independently by a student's t-test, and the significance level was Bonferroni-Holm corrected. *P=0.01, **P=0.002, ***P<0.0001.

FIG. 6. Effect of the high-calorie and low-calorie treatments on liver fat. FIG. 6A shows the percentage of intrahepatic fat, as derived from the percentage of the fat weight in the wet liver weight. The HC treatment induced substantial hepatic steatosis, significantly higher than observed in the lean LC livers (Student t-test, P<0.0001). FIG. 6B shows representative pictures of liver tissue slices from the HC and LC groups analyzed by histopathology with hematoxylin and eosin (H&E) staining. The HC hepatocytes show macro-vesicular steatosis around both the portal (bottom left) and the central (bottom right) veins. The top LC liver slices show no lipid droplets formation.

FIG. 7. Percentage of liver glycogen in the high- and low-calorie diet groups. The HC group had higher values though not statistically significant (student t-test, P=0.13; SEM=0.07).

FIG. 8. Blood glucose levels as a function of time in the different groups; low calorie (LC), high calorie (HC) and treated HC (THC). Values are expressed as the mean±standard error (n=12 for each group). Data were analyzed using two-way ANOVA with repeated measures. Effects of Treatment: P<0.0001, Time: P<0.0001, Treatment x time: P=0.003. Animal effect: P=0.0003. Differences between LC and HC or THC were always statistically significant. * Denotes significant differences (P<0.05) between THC and HC at specific time points by contrast t-tests.

FIG. 9. Body weight in the low calorie (LC), high calorie (HC) and thiamine-treated HC (THC) growing lambs. Repeated measures ANOVA analysis for HC vs. LC revealed an effect of the dietary treatment (P<0.0001), time (P<0.0001) and treatment x time interaction (P<0.0001). Comparing HC with THC revealed an effect of time (P<0.0001) and a treatment x time interaction (P<0.0001) and a trend for thiamine treatment (P=0.09). * Denotes P<0.05 between HC and THC after Bonferroni-Holm correction.

FIG. 10. Daily average dietary energy intake, expressed as metabolizable energy per animal, as a function of time in the low calorie (LC) high calorie (HC) and thiamine-treated HC (THC) groups.

FIG. 11A shows the HC treatment promoted substantial hepatic-fat accumulation that was ameliorated by the thiamine treatment (One-way ANOVA, P<0.0001). Similarly, contrast t-tests between HC and LC as well as between HC and THC were both highly significant (P<0.0001). FIG. 11B shows representative liver pictures and corresponding tissue sections were analyzed by histopathology with H&E staining taken at a ×200 magnification. Macro-vesicular steatosis was observed in the HC lambs, but micro-vesicular steatosis was more consistently present, mainly around lobular zone 3 and occasionally in zone 1. Arrows indicate macro-vesicular steatosis Where the nuclei of the hepatocytes are displaced to the side by large lipid droplets. FIG. 11C shows the hepatic index was computed as the percentage of liver weight per animal body weight. One-way ANOVA detects an effect of treatment (P=0.0002). * Denotes P<0.05 by contrast t-tests. FIG. 11D shows the mosaic frequency plot of the hepatocellular steatosis (the average score of macro-vesicular and micro-vesicular steatosis) graded on a scale from 0 to 3. Differences between the treatments were significant for both the Pearson and the likelihood ratio χ squares (P<0.0001).

FIG. 12. Hepatic glycogen content in the low (LC), high (HC) and thiamine-treated HC (THC) animals. Data analysis by one-way ANOVA detects an effect of Treatment (P=0.001). * Denotes P<0.05 by contrast t-tests.

FIG. 13. Molecular mechanism associated with the dietary and thiamine treatments. Comparative liver expression of genes involved in fatty acid oxidation (FIG. 13A), lipogenesis (FIG. 13B), lipid droplet metabolism and VLDL secretion (FIG. 13C) and, fatty acid uptake (FIG. 13D); in the low-calorie (LC), high-calorie (HC) and thiamine-treated (THC) sheep. Effect of treatment by one-way ANOVA: SIRT1 (P 0.17), PRKAA2 (P=0.002), PPARGC1A (P=0.03), PPARA (P 0.009), AGPAT1 (P=0.1), AGPAT2 (P=0.04), SREBF1 (P=0.22), PPARG (P<0.05), FASN (P=0.02), FOXO1 (P=0.56), Perilipin 2 (P<0.0001), MTTP (P=0.03), APOB (P=0.76), CD36 (P=0.46), SLC27A6 (P=0.003), and SLC27A5 (P=0.04). * Denotes P<0.05 by contrast t-tests. Thiamine appears to destabilize fat storage in lipid droplets and to enhance its export out of the liver with very-low-density lipoprotein (VLDL) particles.

FIG. 14. Comparative expression of proinflammatory genes measured in leukocyte (FIG. 144), liver (FIG. 14B), and of liver endogenous antioxidants (FIG. 14C). One-way ANOVA detected an effect of treatment for leukocyte CCL2 (P=0.04) and CXCL8 (P=0.04); and hepatic TAT (P=0.01), PTX3 (P=0.02), Catalase (P=0.008), and SOD2 (P 0.02). * Denotes P<0.05 by contrast t-tests. The HC diet seems to generally increase the inflammatory state, which was partially alleviated by thiamine.

### DETAILED DESCRIPTION OF THE DISCLOSURE

The present disclosure provides herein a nutritional model for in-vivo fatty liver (FL) research in sheep, using high-calorie (HC) and readily digestible carbohydrate-based diet. Briefly, it was found that growing lambs on the HC diet at limited physical activity for four months induces substantial hepatic steatosis, with abnormal liver-fat content that is twice greater than that of normal livers of lambs grown on a low-calorie (LC) diet (8.1% vs. 5.1%; P<0.0001). Moreover, in addition to gaining significantly more weight than lambs on the LC diet, the HC lambs developed substantial hyperglycemia, hyperinsulinemia, hypertriglyceridemia, systemic and adipose-specific insulin resistance, as well as increased leukocytes expression of inflammatory markers, thereby exhibiting obesity and MetS hallmarks. The inventors recently published a detailed analysis of these data (Kalyesubula et al, Scientific Reports, 2020).

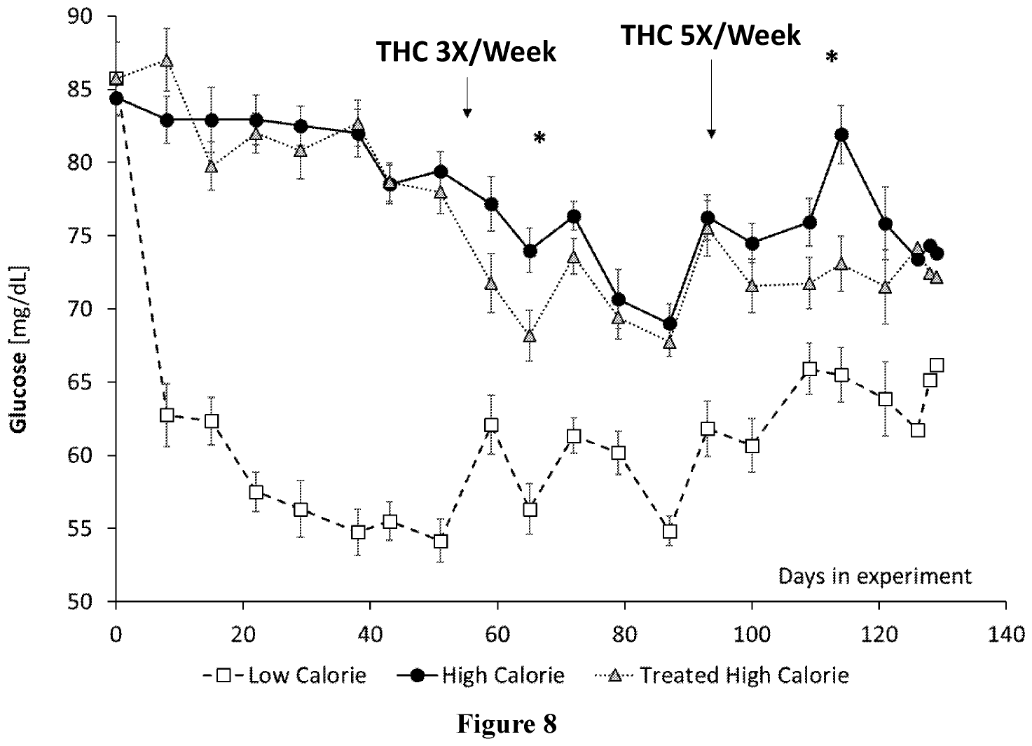

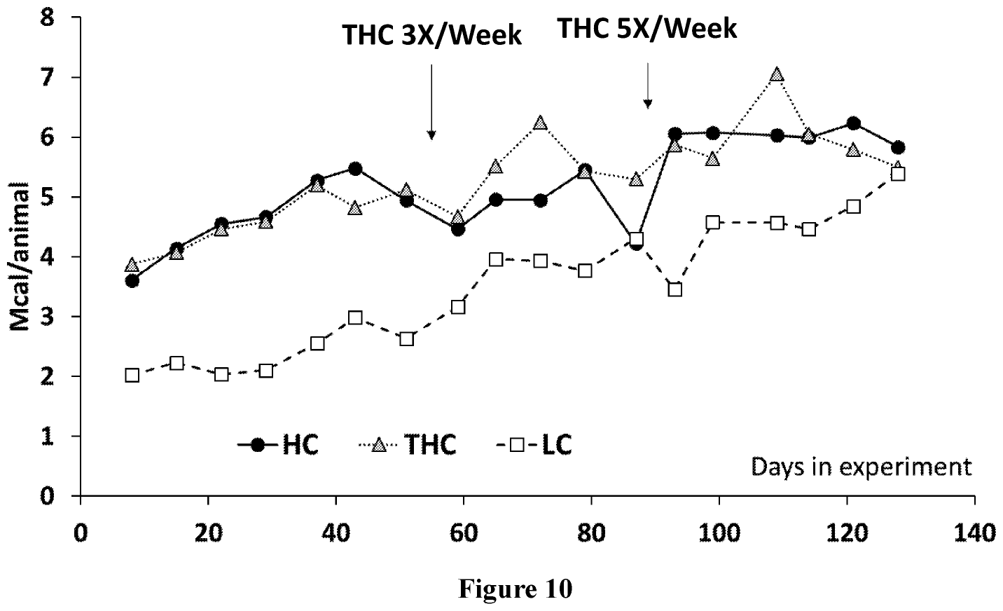

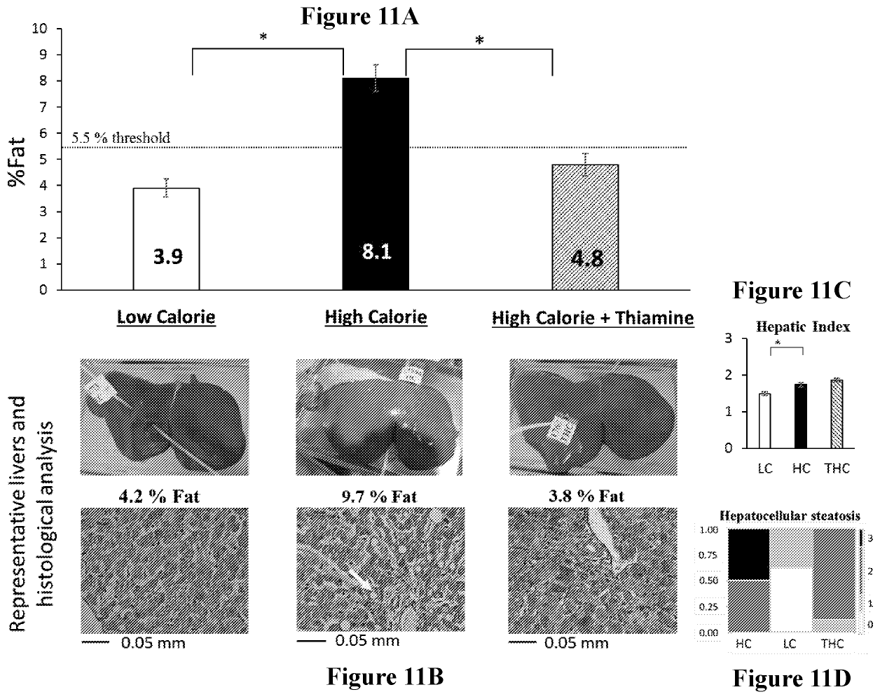

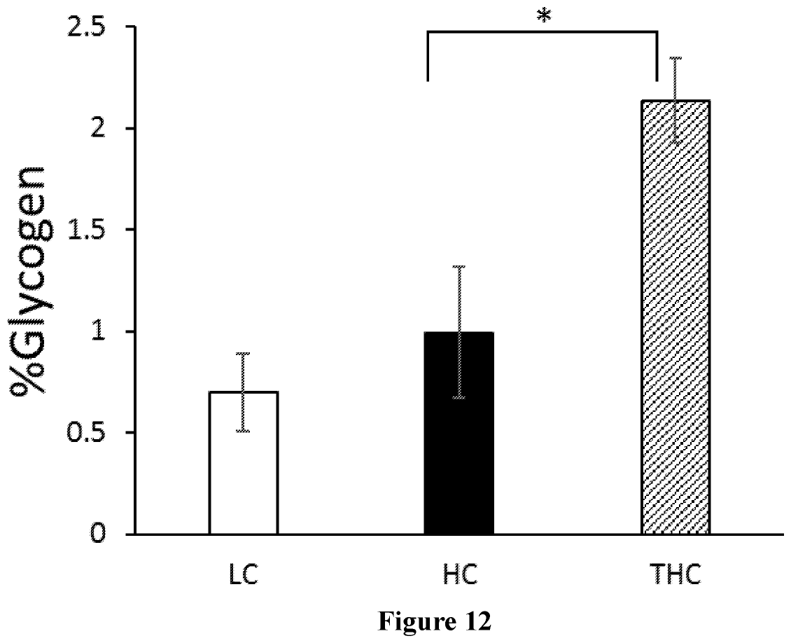

Using this model, it was found that thiamine (Vitamin B1), an important factor for the TCA cycle turnover, has the capacity to reduce hepatic steatosis in obese lambs grown on the HC diet. Weaned lambs (n=36) were randomly assigned to three different growth regimes: LC diet (lean-livers control), HC diet, and HC diet+thiamine treatment (THC). The average caloric intake between the HC and THC groups was indifferent (FIG. 10). Yet, thiamine-treated animals exhibited reduced blood glucose (P=0.02, FIG. 8) and increased liver glycogen (P=0.001, FIG. 12). More, thiamine reduced hepatic steatosis profoundly (HC=8.1% vs. THC=4.8%, P<0.0001), to levels below the 5.5% biochemical threshold for steatosis and statistically indifferent from those in the lean livers of the LC group (3.8%) (FIG. 11A).

These data open new horizons of significant clinical implications in the development of NAFLD therapy using thiamine, thiamine esters, or thiamine analogs, such as Benfotiamine (a synthetic S-acyl derivative of thiamine), Sulbutiamine (a synthetic derivative of thiamine) and others, in the prevention and treatment of hepatic steatosis for NAFLD, alcoholic liver disease obesity, T2D and MetS patients. The added benefit of thiamine to glycemic control makes it particularly attractive for treatment of T2D patients presenting with hepatic steatosis. Moreover, combination therapies using thiamine, thiamine esters, or thiamine analogs, to combat hepatic steatosis, and additional agents to target other aspects of the disease such as insulin resistance, liver oxidative stress and inflammation, using for instance, insulin sensitizers and/or vitamin E, respectively, may provide a more effective approach to treat this multifaceted disease. Moreover, thiamine, thiamine esters, or thiamine analogs, may be used together with a calorie-restricted diet and physical exercise to more efficiently target NAFLD.

Apart from the benefits of using a large animal as a preclinical model system of similar weight and size to human, one of the main advantage of the particular fatty liver model in sheep is based in its spontaneous development (no genetic mutations needed) with overnutrition based on carbohydrates abundance, rather than the common high-fat or high-cholesterol diets used in other models. This reflects not only the typical nutrition leading to MetS, but also key metabolic derangements such as insulin resistance hyperglycemia and dyslipidemia typified by MetS.

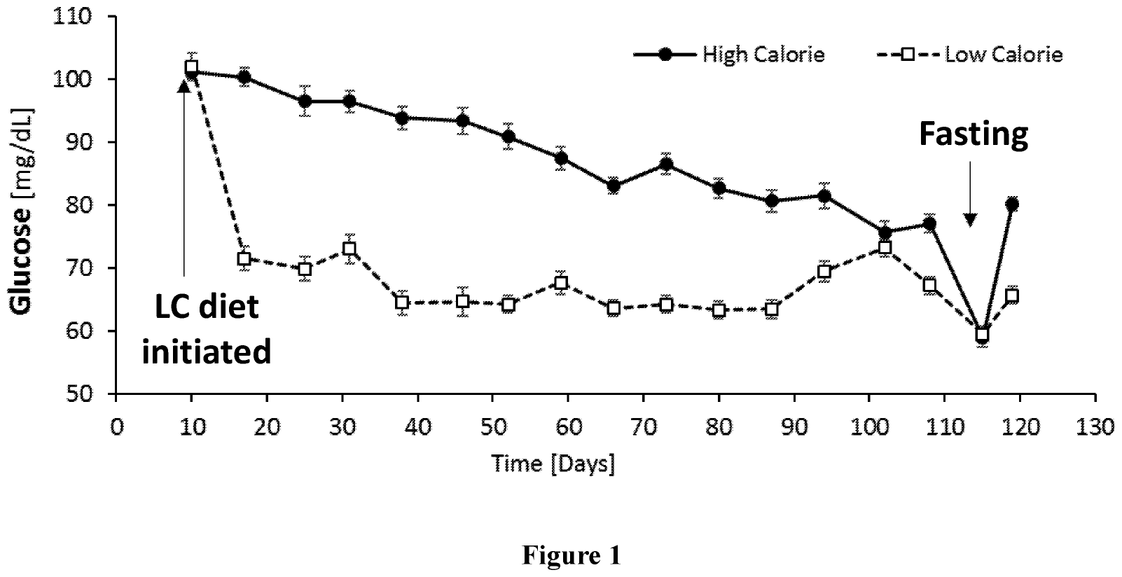

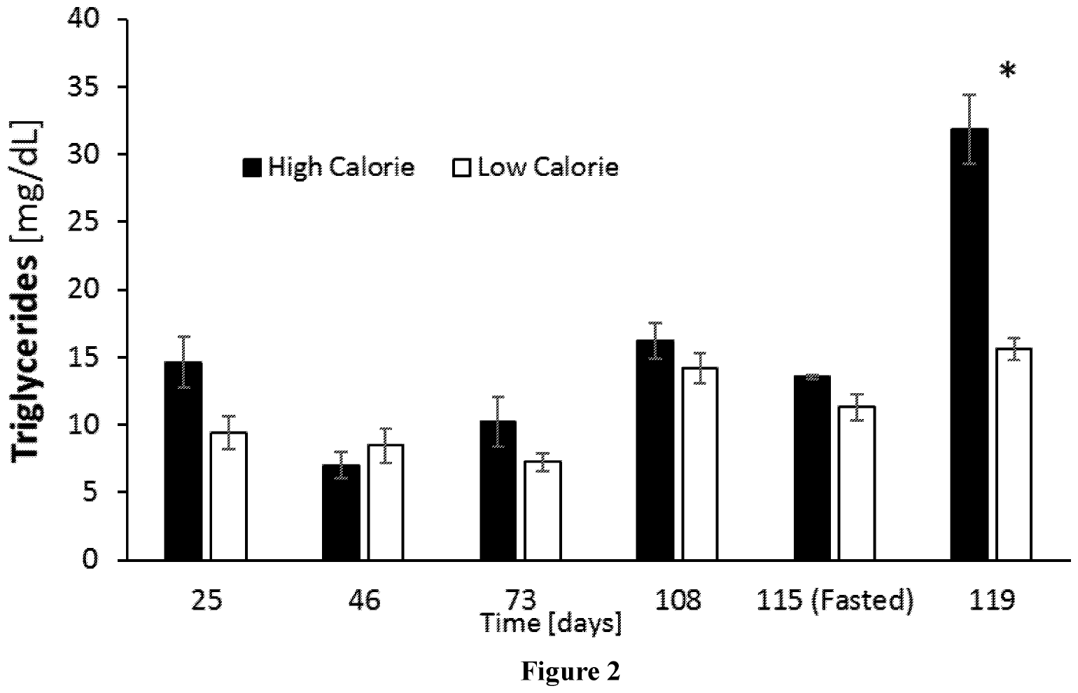

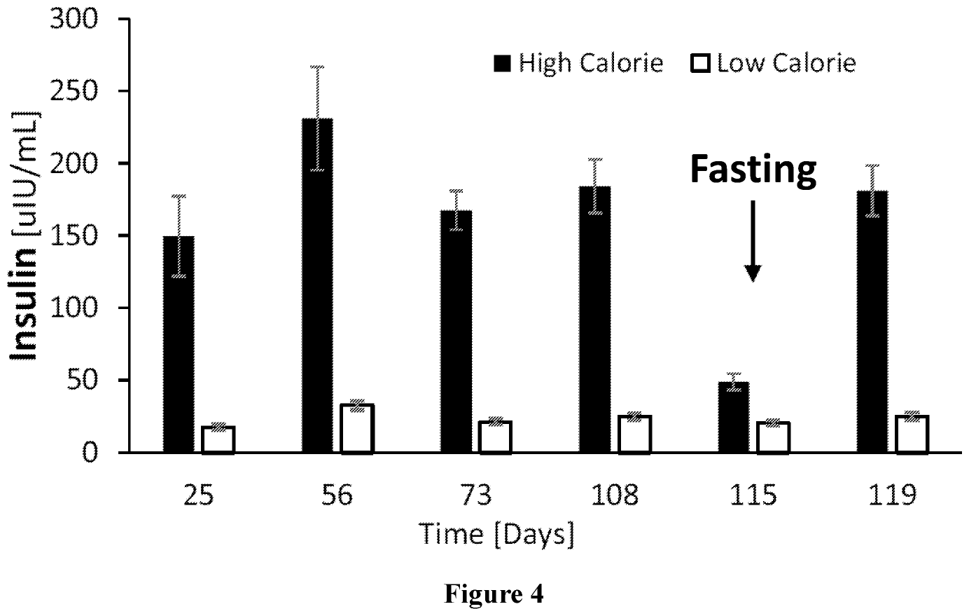

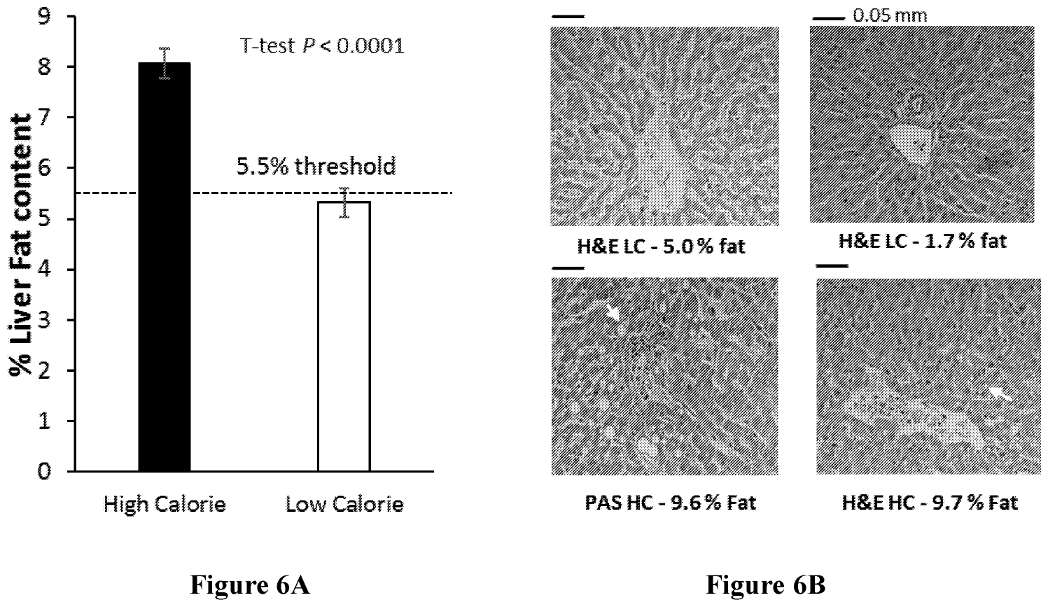

More specific and quantitative characteristics of the sheep model are: lambs grown on the HC diet were consistently more hyperglycemic (FIG. 1) and hyper-insulinemic (FIG. 2) than lambs grown on the lower-calorie (LC) diet (P<0.0001). As a result, the HC lambs developed systemic- (HOMA-IR of 7.3 vs. 3.1; P<0.0001), and adipose- (ADIPO-IR of 342.7 vs. 74.4; P<0.0001) insulin resistance, significant adiposity as measured by BMI and body condition score (P<0.0001), and higher plasma triglycerides (P<0.05) (FIG. 4). Circulating leukocytes in the HC lambs had higher mRNA expression levels of the proinflammatory markers CCL2 (P=0.01) and TNF-alpha (P=0.04), and IL1B trended higher (P<0.1). Remarkably, lambs on the HC diet developed substantial liver steatosis (mean fat content of 8.1 vs. 5.1% in the LC group, P<0.0001) with a higher histological steatosis score (2.1 vs. 0.4, P=0.0002, FIG. 6).

This model offers additional practical experimental advantages, including long lifespan for extended follow-up durations (years), large quantities of blood and liver samples that can be collected repeatedly at a relatively high frequency for more accurate analysis and quantitation.

The complexity of the carbohydrates in the diet of ruminants determines the level of glucose precursors and metabolizable energy that can be available to the animal. In growing lambs, it is well reflected in their blood glucose levels. Thus, by feeding lambs with rations of varying consumed metabolizable energy, two populations of significantly different glycemic indexes were obtained. The over-nourished lambs were consistently hyperglycemic and hyper-insulinemic, and in four months, developed adiposity, dyslipidemia, insulin resistance, significant hepatic steatosis and hepatomegaly. Hyperglycemia, representing the high-carbohydrates abundance, was the strongest predictor of hepatic steatosis. Surprisingly, steatosis was negatively correlated with circulating NEFA, suggesting that hepatic DNL may play a more significant role than adipose lipolysis in the initiation of steatosis. The systemic metabolic and liver abnormalities induced by the HC diet in sheep are similar to those observed in humans with MetS and liver steatosis; therefore, this large-animal model is of high value to NAFLD research and therapy development (Kalyesubula et al., Scientific Reports, 2020).

The present invention provides, in one aspect, a method of preventing or treating a condition selected from the group consisting of: (i) fatty liver (FL) disease (FLD), (ii) a FL-associated disease, and (iii) a FL-associated symptom, in a patient in need of such prevention or treatment, the method comprising the step of administering a therapeutically effective amount of thiamine, a thiamine ester, or a thiamine analog, to the patient.

In certain embodiments, the condition is FLD.

In certain embodiments, the condition is a FL-associated disease selected from the group consisting of non-alcoholic fatty liver disease (NAFLD), alcoholic fatty liver disease, non-alcoholic steatohepatitis (NASH), metabolic syndrome (MetS), type-2 diabetes mellitus (T2D), cardiovascular disease, dyslipidemia, alcoholic liver disease, hepatocellular carcinoma (HCC), viral steatohepatitis, overnutrition, overweight, and obesity.

In certain embodiments, the condition is NAFLD. In certain embodiments, the condition is alcoholic fatty liver disease. In certain embodiments, the condition is NASH. In certain embodiments, the condition is MetS. In certain embodiments, the condition is T2D. In certain embodiments, the condition is cardiovascular disease. In certain embodiments, the condition is dyslipidemia. In certain embodiments, the condition is alcoholic liver disease. In certain embodiments, the condition is HCC. In certain embodiments, the condition is viral steatohepatitis. In certain embodiments, the condition is overnutrition. In certain embodiments, the condition is overweight. In certain embodiments, the condition is obesity.

In certain embodiments, the condition is a FL-associated symptom selected from the group consisting of hepatic steatosis, hepatic cirrhosis, hepatic inflammation, hepatic fibrosis, hepatic pain, hepatic yellow pigmentation, decreased liver function, low liver glycogen level, tiredness, systemic insulin resistance, hyperglycemia, systemic hypertriglyceridemia, micro-vesicular steatosis, macro-vesicular steatosis, high food intake, and high weight gain.

In certain embodiments, the condition is hepatic steatosis. In certain embodiments, the condition is hepatic cirrhosis. In certain embodiments, the condition is hepatic inflammation. In certain embodiments, the condition is hepatic fibrosis. In certain embodiments, the condition is hepatic pain. In certain embodiments, the condition is hepatic yellow pigmentation. In certain embodiments, the condition is decreased liver function. In certain embodiments, the condition is low liver glycogen level. In certain embodiments, the condition is tiredness. In certain embodiments, the condition is systemic insulin resistance. In certain embodiments, the condition is hyperglycemia. In certain embodiments, the condition is systemic hypertriglyceridemia. In certain embodiments, the condition is micro-vesicular steatosis. In certain embodiments, the condition is macro-vesicular steatosis. In certain embodiments, the condition is high food intake. In certain embodiments, the condition is high weight gain.

In certain embodiments, the FL-associated symptom is selected from the group consisting of insulin resistance, hepatic steatosis, dyslipidemia, hyperglycemia, cirrhosis, hepatic inflammation, hepatic fibrosis, decreased liver function, low liver glycogen level, high food intake, and weight gain. In certain embodiments, the FL-associated symptom is insulin resistance. In certain embodiments, the FL-associated symptom is hepatic steatosis. In certain embodiments, the FL-associated symptom is dyslipidemia. In certain embodiments, the FL-associated symptom is hyperglycemia. In certain embodiments, the FL-associated symptom is cirrhosis. In certain embodiments, the FL-associated symptom is hepatic inflammation. In certain embodiments, the FL-associated symptom is hepatic fibrosis. In certain embodiments, the FL-associated symptom is decreased liver function. In certain embodiments, the FL-associated symptom is a low liver glycogen level. In certain embodiments, the FL-associated symptom is high food intake. In certain embodiments, the FL-associated symptom is weight gain.

In one embodiment, the method is for preventing or treating fatty liver (FL). In one embodiment, the method is for preventing or treating a FL-associated disease. In one embodiment, the method is for preventing or treating a FL-associated symptom. In one embodiment, the method is for preventing or treating overweight. In one embodiment, the method is for preventing or treating obesity. In one embodiment, the method is for preventing or treating FL-unassociated overweight. In one embodiment, the method is for preventing or treating FL-unassociated obesity.

In certain embodiments, the FL-associated disease is selected from the group consisting of overweight, obesity, non-alcoholic FL disease (NAFLD), metabolic syndrome (MetS), type-2 diabetes mellitus (T2D), cardiovascular disease, dyslipidemia, and alcoholic liver disease. In certain embodiments, the FL-associated disease is obesity or MetS or T2D or alcoholic liver disease. In certain embodiments, the FL-associated disease is overweight. In certain embodiments, the FL-associated disease is obesity. In certain embodiments, the FL-associated disease is a non-alcoholic FL disease (NAFLD). In certain embodiments, the FL-associated disease is metabolic syndrome (MetS). In certain embodiments, the FL-associated disease is type-2 diabetes mellitus (T2D). In certain embodiments, the FL-associated disease is cardiovascular disease. In certain embodiments, the FL-associated disease is dyslipidemia. In certain embodiments, the FL-associated disease is alcoholic liver disease.

In certain embodiments, the hepatic steatosis is at least 5% by weight fat content (wet/wet). In certain embodiments, the hepatic steatosis is at least 5.5% by weight fat content (wet/wet). In certain embodiments, the hepatic steatosis is at least 6% by weight fat content (wet/wet). In certain embodiments, the hepatic steatosis is at least 7% by weight fat content (wet/wet). In certain embodiments, the hepatic steatosis is at least 8% by weight fat content (wet/wet).

In certain embodiments, the hepatic steatosis is 5% by weight fat content (wet/wet). In certain embodiments, the hepatic steatosis is 5.5% by weight fat content (wet/wet). In certain embodiments, the hepatic steatosis is 6% by weight fat content (wet/wet). In certain embodiments, the hepatic steatosis is 7% by weight fat content (wet/wet). In certain embodiments, the hepatic steatosis is 8% by weight fat content (wet/wet).

In certain embodiments, the hepatic steatosis is 5% to 8% by weight fat content (wet/wet). In certain embodiments, the hepatic steatosis is 5.5% to 8% by weight fat content (wet/wet). In certain embodiments, the hepatic steatosis is 6% to 8% by weight fat content (wet/wet). In certain embodiments, the hepatic steatosis is 7% to 8% by weight fat content (wet/wet). In certain embodiments, the hepatic steatosis is 8% to 9% by weight fat content (wet/wet). In certain embodiments, the hepatic steatosis is 9% to 10% by weight fat content (wet/wet).

In certain embodiments, the patient is afflicted with Type-2 diabetes mellitus (T2D) and/or insulin resistance. In certain embodiments, the patient is afflicted with T2D. In certain embodiments, the patient is afflicted with insulin resistance. In certain embodiments, the patient is afflicted with T2D and insulin resistance. In certain embodiments, the patient is afflicted with T2D but not with insulin resistance. In certain embodiments, the patient is afflicted with insulin resistance but not with T2D.

In certain embodiments, the patient is not afflicted with Type-2 diabetes mellitus (T2D) and/or insulin resistance.

In certain embodiments, the patient is not afflicted with T2D. In certain embodiments, the patient is not afflicted with insulin resistance. In certain embodiments, the patient is not afflicted with T2D and is not afflicted with insulin resistance.

As would be understood by a person of the art, according to the principles of the present invention, in certain embodiments, when specifying doses of thiamine esters or thiamine analogs, these doses may be altered to be doses of thiamine esters or thiamine analogs which are of equivalent pharmacologic activity to that of thiamine at the specified dose.

In certain embodiments, the method comprises administering about 100 mg thiamine, a thiamine ester, or a thiamine analog. In certain embodiments, the method comprises administering about 200 mg thiamine, a thiamine ester, or a thiamine analog. In certain embodiments, the method comprises administering about 300 mg thiamine, a thiamine ester, or a thiamine analog. In certain embodiments, the method comprises administering about 400 mg thiamine, a thiamine ester, or a thiamine analog. In certain embodiments, the method comprises administering about 500 mg thiamine, a thiamine ester, or a thiamine analog. In certain embodiments, the method comprises administering about 600 mg thiamine, a thiamine ester, or a thiamine analog. In certain embodiments, the method comprises administering about 700 mg thiamine, a thiamine ester, or a thiamine analog. In certain embodiments, the method comprises administering about 800 mg thiamine, a thiamine ester, or a thiamine analog. In certain embodiments, the method comprises administering about 900 mg thiamine, a thiamine ester, or a thiamine analog. In certain embodiments, the method comprises administering about 1000 mg thiamine, a thiamine ester, or a thiamine analog.

In certain embodiments, the method comprises administering about 100 mg thiamine, a thiamine ester, or a thiamine analog, to about 1000 mg thiamine, a thiamine ester, or a thiamine analog, every day. In certain embodiments, the method comprises administering about 200 mg thiamine, a thiamine ester, or a thiamine analog, to about 500 mg thiamine, a thiamine ester, or a thiamine analog, every day. In certain embodiments, the method comprises administering about 300 mg thiamine, a thiamine ester, or a thiamine analog, to about 400 mg thiamine, a thiamine ester, or a thiamine analog, every day. In certain embodiments, the method comprises administering about 100 mg thiamine, a thiamine ester, or a thiamine analog, every day. In certain embodiments, the method comprises administering about 200 mg thiamine, a thiamine ester, or a thiamine analog, every day. In certain embodiments, the method comprises administering about 100 mg thiamine, a thiamine ester, or a thiamine analog, every day. In certain embodiments, the method comprises administering about 400 mg thiamine, a thiamine ester, or a thiamine analog, every day. In certain embodiments, the method comprises administering about 500 mg thiamine, a thiamine ester, or a thiamine analog, every day. In certain embodiments, the method comprises administering about 600 mg thiamine, a thiamine ester, or a thiamine analog, every day. In certain embodiments, the method comprises administering about 700 mg thiamine, a thiamine ester, or a thiamine analog, every day. In certain embodiments, the method comprises administering about 800 mg thiamine, a thiamine ester, or a thiamine analog, every day. In certain embodiments, the method comprises administering about 900 mg thiamine, a thiamine ester, or a thiamine analog, every day. In certain embodiments, the method comprises administering about 1000 mg thiamine, a thiamine ester, or a thiamine analog, every day.

In certain embodiments, the method comprises administering about 100 mg thiamine, a thiamine ester, or a thiamine analog, to about 1000 mg thiamine, a thiamine ester, or a thiamine analog. In certain embodiments, the method comprises administering about 200 mg thiamine, a thiamine ester, or a thiamine analog, to about 500 mg thiamine, a thiamine ester, or a thiamine analog. In certain embodiments, the method comprises administering about 300 mg thiamine, a thiamine ester, or a thiamine analog, to about 400 mg thiamine, a thiamine ester, or a thiamine analog. In certain embodiments, the method comprises administering about 100 mg thiamine, a thiamine ester, or a thiamine analog. In certain embodiments, the method comprises administering about 200 mg thiamine, a thiamine ester, or a thiamine analog. In certain embodiments, the method comprises administering about 300 mg thiamine, a thiamine ester, or a thiamine analog. In certain embodiments, the method comprises administering about 400 mg thiamine, a thiamine ester, or a thiamine analog. In certain embodiments, the method comprises administering about 500 mg thiamine, a thiamine ester, or a thiamine analog. In certain embodiments, the method comprises administering about 600 mg thiamine, a thiamine ester, or a thiamine analog. In certain embodiments, the method comprises administering about 700 mg thiamine, a thiamine ester, or a thiamine analog. In certain embodiments, the method comprises administering about 800 mg thiamine, a thiamine ester, or a thiamine analog. In certain embodiments, the method comprises administering about 900 mg thiamine, a thiamine ester, or a thiamine analog. In certain embodiments, the method comprises administering about 1000 mg thiamine, a thiamine ester, or a thiamine analog.

In certain embodiments, the method comprises administering thiamine, a thiamine ester, or a thiamine analog, once every 7 days. In certain embodiments, the method comprises administering thiamine, a thiamine ester, or a thiamine analog, once every 6 days. In certain embodiments, the method comprises administering thiamine, a thiamine ester, or a thiamine analog, once every 5 days. In certain embodiments, the method comprises administering thiamine, a thiamine ester, or a thiamine analog, once every 4 days. In certain embodiments, the method comprises administering thiamine, a thiamine ester, or a thiamine analog, once every 3 days. In certain embodiments, the method comprises administering thiamine, a thiamine ester, or a thiamine analog, once every 2 days. In certain embodiments, the method comprises administering thiamine, a thiamine ester, or a thiamine analog, once every day.

In certain embodiments, the method comprises administering thiamine, a thiamine ester, or a thiamine analog, 3 times every day. In certain embodiments, the method comprises administering thiamine, a thiamine ester, or a thiamine analog, 2 times every day.

In certain embodiments, the method comprises administering about 100 mg thiamine, a thiamine ester, or a thiamine analog, to about 600 mg thiamine, a thiamine ester, or a thiamine analog, every day. In certain embodiments, the method comprises administering about 200 mg thiamine, a thiamine ester, or a thiamine analog, to about 500 mg thiamine, a thiamine ester, or a thiamine analog, every day. In certain embodiments, the method comprises administering about 300 mg thiamine, a thiamine ester, or a thiamine analog, to about 400 mg thiamine, a thiamine ester, or a thiamine analog, every day.

In certain embodiments, the method comprises administering about 100 mg thiamine to about 600 mg thiamine, every day. In certain embodiments, the method comprises administering about 100 mg thiamine ester to about 600 mg thiamine ester, every day. In certain embodiments, the method comprises administering about 100 mg thiamine analog to about 600 mg thiamine analog, every day.

In certain embodiments, the method comprises administering about 200 mg thiamine to about 500 mg thiamine, every day. In certain embodiments, the method comprises administering about 200 mg thiamine ester to about 500 mg thiamine ester, every day. In certain embodiments, the method comprises administering about 200 mg thiamine analog to about 500 mg thiamine analog, every day.

In certain embodiments, the method comprises administering about 300 mg thiamine to about 400 mg thiamine, every day. In certain embodiments, the method comprises administering about 300 mg thiamine ester to about 400 mg thiamine ester, every day. In certain embodiments, the method comprises administering about 300 mg thiamine analog to about 400 mg thiamine analog, every day.

In certain embodiments, the thiamine, a thiamine ester, or a thiamine analog, is administered to the patient by systemic administration. In certain embodiments, the thiamine, a thiamine ester, or a thiamine analog, is administered to the patient by oral administration, intravenous administration or intramuscular administration. In certain embodiments, the thiamine, a thiamine ester, or a thiamine analog, is administered to the patient by oral administration. In certain embodiments, the thiamine, a thiamine ester, or a thiamine analog, is administered to the patient by intravenous administration. In certain embodiments, the thiamine, a thiamine ester, or a thiamine analog, is administered to the patient by intramuscular administration.

In certain embodiments, the thiamine, a thiamine ester, or a thiamine analog, is administered to the patient by oral administration, intravenous administration or intramuscular administration.

In certain embodiments, the thiamine, a thiamine ester, or a thiamine analog, is administered to the patient by oral administration. In certain embodiments, the thiamine, a thiamine ester, or a thiamine analog, is administered to the patient by intravenous administration. In certain embodiments, the thiamine, a thiamine ester, or a thiamine analog, is administered to the patient by intramuscular administration.

In certain embodiments, the method comprises administering thiamine. Thiamine may be identified with CAS Number of 70-16-6, or with CAS Number of 67-03-8 for thiamine hydrochloride.

In certain embodiments, the method comprises administering a thiamine ester. The phrase “thiamine ester” as used herein generally refers to a chemical compound derived from thiamine in which at least one —OH (hydroxyl) group is replaced by an —O-alkyl (alkoxy) group.

In certain embodiments, the thiamine ester is selected from the group consisting of thiamine monophosphate and thiamine pyrophosphate.

In certain embodiments, the thiamine ester is thiamine monophosphate. In certain embodiments, the thiamine ester is thiamine pyrophosphate.

In certain embodiments, the method comprises administering a thiamine analog. The phrase “thiamine analog” as used herein generally refers to analogs or analogues of vitamin B1, thiamine. They typically have improved bioavailability relative to thiamine and are typically used to treat conditions caused by vitamin B1 deficiency, such as beriberi, Korsakoff s syndrome, Wernicke's encephalopathy and diabetic neuropathy.

In certain embodiments, the thiamine analog is selected from the group consisting of Acefurtiamine, Acetiamine, Allithiamine, Beclotiamine, Benfotiamine, Bentiamine, Bisbentiamine, Cetotiamine, Cycotiamine, Fursultiamine, Monophosphothiamine, Octotiamine, Prosultiamine, Sulbutiamine, and Vintiamol.

In certain embodiments, the thiamine analog is Acefurtiamine. In certain embodiments, the thiamine analog is Acetiamine. In certain embodiments, the thiamine analog is Allithiamine. In certain embodiments, the thiamine analog is Beclotiamine. In certain embodiments, the thiamine analog is Benfotiamine. In certain embodiments, the thiamine analog is Bentiamine. In certain embodiments, the thiamine analog is Bisbentiamine. In certain embodiments, the thiamine analog is Cetotiamine. In certain embodiments, the thiamine analog is Cycotiamine. In certain embodiments, the thiamine analog is Fursultiamine. In certain embodiments, the thiamine analog is Monophosphothiamine. In certain embodiments, the thiamine analog is Octotiamine. In certain embodiments, the thiamine analog is Prosultiamine. In certain embodiments, the thiamine analog is Sulbutiamine. In certain embodiments, the thiamine analog is Vintiamol.

In certain embodiments, the method further comprises increasing the amount or intensity of physical exercise of the patient. In certain embodiments, the method further comprises increasing the amount of physical exercise of the patient. In certain embodiments, the method further comprises increasing the intensity of physical exercise of the patient. In certain embodiments, the method further comprises increasing the amount and intensity of physical exercise of the patient.

In certain embodiments, the method comprises changing the diet of the patient to a calorie-restricted diet. As would be understood by a person of the art, the term “calorie-restricted diet” refers to a dietary regimen based on a reduced calorie intake, which, for example, may be lower than a patient's previous intake, i.e. before intentionally restricting calories. A reduction of daily calorie intake by about 500 kcal usually is regarded as a “moderate calorie restriction” or “moderate energy restriction”.

In certain embodiments, the method comprises changing the diet of the patient to a calorie-restricted diet and increasing the amount or intensity of physical exercise of the patient.

In certain embodiments, the patient is a human.

In certain embodiments, the patient is a ruminant animal, and the FL-associated disease is ketosis and pregnancy toxemia. In certain embodiments, the ruminant animal is selected from the group consisting of domestic cows, sheep and goats.

In certain embodiments, the patient is a domestic cat, and the FL-associated disease is selected from the group consisting of fatty liver disease and hepatic lipidosis.

The present invention further provides, in another aspect, a composition comprising thiamine, a thiamine ester, or a thiamine analog, and at least one additional agent selected from the group consisting of an insulin sensitizer, an insulin-releasing enhancer, and an antioxidant.

The phrase “insulin sensitizer” as used herein generally refers to drugs (molecules) which lower blood sugar levels by increasing muscle, fat and/or liver sensitivity to insulin.

In certain embodiments, the insulin sensitizer is selected from the group consisting of a Biguanide, a Thiazolidinedione, and a Lyn kinase activator.

In certain embodiments, the insulin sensitizer is a Biguanide. The term “Biguanide” or “Biguanidine” as interchangeably used herein generally refers to a class of drugs that function as oral antihyperglycemic drugs used for diabetes mellitus or prediabetes treatment. In certain embodiments, the Biguanide is Metformin.

In certain embodiments, the insulin sensitizer is a Thiazolidinedione. The term “Thiazolidinedione” or “TZD” as interchangeably used herein generally refers to a class of heterocyclic compounds consisting of a five-membered C3NS ring. The term includes a family of drugs used in the treatment of diabetes mellitus type 2. Thiazolidinediones or TZDs act by activating PPARs (peroxisome proliferator-activated receptors), a group of nuclear receptors, specific for PPARγ (PPAR-gamma, PPARG), and are thus the PPARG agonists subset of PPAR agonists. In certain embodiments, the Thiazolidinedione is Pioglitazone. In certain embodiments, the Thiazolidinedione is Lobeglitazone.

In certain embodiments, the insulin sensitizer is a Lyn kinase activator. Tyrosine-protein kinase Lyn is a protein that in humans is encoded in humans by the LYN gene. The phrase “Lyn kinase activator” as used herein generally refers to drugs (molecules) which activate the Tyrosine-protein kinase Lyn. In certain embodiments, the Lyn kinase activator is Tolimidone (MLR-1023).

The phrase “insulin-releasing enhancer” as used herein generally refers to drugs (molecules) which increase the production and/or release of insulin from insulin-producing cells.

In certain embodiments, the insulin-releasing enhancer is a glucagon-like peptide-1 receptor (GLP-1 receptor) agonist. The phrase “GLP-1 receptor agonist” or “incretin mimetics” as interchangeably used herein generally refers to agonists of the GLP-1 receptor. This class of medications may be used for the treatment of type 2 diabetes. As GLP-1 as has a short duration of action on its receptor, GLP-1 receptor agonists may have one or several modifications to overcome this limitation. In certain embodiments, the GLP-1 receptor agonist is Liraglutide. In certain embodiments, the GLP-1 receptor agonist is Exenatide. In certain embodiments, the GLP-1 receptor agonist is Lixisenatide. In certain embodiments, the GLP-1 receptor agonist is Albiglutide. In certain embodiments, the GLP-1 receptor agonist is Dulaglutide. In certain embodiments, the GLP-1 receptor agonist is Semaglutide.

The phrase “antioxidant” as used herein generally refers to drugs (molecules) which inhibit oxidation, a chemical reaction that can produce free radicals, thereby leading to chain reactions that may damage the cells of organisms.

In certain embodiments, the antioxidant is selected from the group consisting of vitamin E, vitamin C, glutathione, lipoic acid, uric acid, (3-carotene, vitamin A, and ubiquinol. In certain embodiments, the antioxidant is vitamin E. In certain embodiments, the antioxidant is vitamin C. In certain embodiments, the antioxidant is glutathione. In certain embodiments, the antioxidant is lipoic acid. In certain embodiments, the antioxidant is uric acid. In certain embodiments, the antioxidant is (3-carotene. In certain embodiments, the antioxidant is vitamin A. In certain embodiments, the antioxidant is ubiquinol.

The present invention further provides, in another aspect, a dosage form, comprising any one of the compositions described above.

The phrase “dosage form” or “unit dose” as used herein generally refers to pharmaceutical or veterinary drug products in the form in which they are marketed for use, with a specific mixture of active ingredients and inactive components (excipients), in a particular configuration (such as a capsule), and apportioned into a particular dose.

The present invention further provides, in another aspect, a method for inducing fatty liver (FL), a FL-associated disease, or a FL-associated symptom, in a large animal, comprising the step of administering a high-calorie diet to the animal.

In certain embodiments, the animal is a ruminant mammal. In certain embodiments, the animal is a sheep. In certain embodiments, the sheep is a domesticates sheep. In certain embodiments, the sheep is Ovis aries.

In certain embodiments, the high-calorie diet comprises an average daily metabolizable energy of over 3.5 MCal. In certain embodiments, the high-calorie diet comprises an average daily metabolizable energy of over 4 MCal. In certain embodiments, the high-calorie diet comprises an average daily metabolizable energy of over 4.5 MCal. In certain embodiments, the high-calorie diet comprises an average daily metabolizable energy of over 5 MCal. In certain embodiments, the high-calorie diet comprises an average daily metabolizable energy of over 5.5 MCal. In certain embodiments, the high-calorie diet comprises an average daily metabolizable energy of over 6 MCal. In certain embodiments, the high-calorie diet comprises an average daily metabolizable energy of over 6.5 MCal.

In certain embodiments, the high-calorie diet comprises an average daily metabolizable energy of 4 MCal. In certain embodiments, the high-calorie diet comprises an average daily metabolizable energy of 4.5 MCal. In certain embodiments, the high-calorie diet comprises an average daily metabolizable energy of 5 MCal. In certain embodiments, the high-calorie diet comprises an average daily metabolizable energy of 5.5 MCal. In certain embodiments, the high-calorie diet comprises an average daily metabolizable energy of 6 MCal. In certain embodiments, the high-calorie diet comprises an average daily metabolizable energy of 6.3 MCal. In certain embodiments, the high-calorie diet comprises an average daily metabolizable energy of 6.5 MCal.

In certain embodiments, the method further comprises administering a drug to the animal.

The term “drug” as used herein generally refers to a chemical substance, of known structure, which, when administered to a living organism, produces a biological effect. A pharmaceutical drug, also called a medication or medicine, is a chemical substance used to treat, cure, prevent, or diagnose a disease or to promote well-being.

In certain embodiment, the drug is an experimental drug, administered to test its effect on fatty liver (FL), a FL-associated disease, or a FL-associated symptom.

In certain embodiments, the method further comprises testing the effect of the drug on the fatty liver (FL), the FL-associated disease, or the FL-associated symptom, in the animal.

In certain embodiments, testing the effect of the drug on the fatty liver (FL), the FL-associated disease, or the FL-associated symptom, in the animal comprises (i) determining a pre-drug-administration state in the animal, (ii) administering the drug to the animal, and (iii) determining a post-drug-administration state in the animal.

As would be understood by a person of the art, the phrase “preventing or treating” as used herein refers to inhibiting or arresting the development of a disease, disorder or condition and/or causing the reduction, remission, or regression of a disease, disorder or condition or keeping a disease, disorder or medical condition from occurring in a subject who may be at risk for the disease disorder or condition, but has not yet been diagnosed as having the disease disorder or condition. Those of skill in the art will understand that various methodologies and assays can be used to assess the development of a disease, disorder or condition, and similarly, various methodologies and assays may be used to assess the reduction, remission or regression of a disease, disorder or condition.

As would be understood by a person of the art, the term “patient” as used herein refers to an animal, preferably a mammal, most preferably a human being, including both young and old human beings of both genders who suffer from or are predisposed to fatty liver disease or condition.

As used herein, the term “about” refers to a range of ±10% of an indicated value.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “an agent” or “at least one agent” may include a plurality of agents, including mixtures thereof.

Throughout this application, various embodiments of the disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 100 to 1000 should be considered to have specifically disclosed subranges such as from 100 to 300, from 100 to 400, from 100 to 500, from 200 to 400, from 200 to 600, from 300 to 600 etc., as well as individual numbers within that range, for example, 100, 200, 300, 400, 500, and 600. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

### EXAMPLES

Methods.

Animals and Experimental Design.

All procedures involving animals in this study were approved by the Volcani Center Animal Care Committee (permits #674/18IL, 790/18IL). The experiments were conducted at the Volcani sheep experimental sheep pen, located in Rishon LeZion, Israel. In the first experiment, which was undertaken to establish a model for hepatic steatosis in sheep, thirty-one male lambs of the Afec-Assaf breed (Gootwine et al., 2008) (2.2±0.03 months old, 27±0.85 Kg in body weight) were randomly assigned to two treatment groups. In the first treatment, lambs were reared on a high calorie (HC) diet (n=15), and the second control treatment was based on a low calorie (LC) diet (n=16). The HC animals were offered ad libitum access to concentrate pellets supplemented with oat hay to constitute 8% of the ration. The LC animals were offered ad libitum access to oat hay supplemented with 14% corn and 13% soy. The left-over feeds were weighed weekly to estimate intake. The intakes were adjusted to meet the body requirements for growth at each stage. The HC animals had an average daily metabolizable energy of 6.3 MCal whereas the LC animals had an average daily metabolizable energy of 3.5 MCal. Both diets contained sufficient vitamins and minerals for growth.

In the second experiment, which was designed to test the effect of thiamine on hepatic steatosis, thirty-six 2-month old male lambs were randomly assigned to three treatment groups (n=12 each). Lambs in the first group were raised on the LC diet, in the second on the HC diet, and in third on the HC diet+300 mg/day of thiamine (initially 3, then 5 days a week) injected subcutaneously. Subcutaneous thiamine injections (300 mg/animal dissolved in 2 mL of filter-sterilized 0.9% saline solution) were administered three times a week from day 50, and then on day 93 the weekly dose was increased by injecting five times a week to account for the increase in body weights. The LC and HC groups were injected similarly with an equal volume of saline. The animals were provided with ad libitum of their respective ration, with free access to fresh drinking water. The group feeds and left-overs were weighed weekly. The group means of daily energy intake were 5.16, 5.29, and 3.50 MCal of metabolizable energy for the HC, THC, and LC, respectively. These dietary treatments were provided for the entire experimental duration (135 days), whereas thiamine treatment was given only during the last 85 days.

Differences between HC and LC lambs were attributed to the dietary effect, whereas differences between THC and HC to the effect of the thiamine treatment.

Blood, Plasma and Liver Sampling.

Weekly, blood glucose concentrations were monitored using the FreeStyle Optium glucometer (Abbot Diabetes Care Ltd., Oxfordshire, UK) (Pichler et al., 2014), and likewise, body weight measurements were taken to determine weight gain. Monthly, blood samples of 5 mL were collected from each animal via venipuncture of the jugular vein into heparinized vacutainers and immediately placed on ice (Vacutainer; Becton Dickinson and Co., Franklin Lakes, N.J.), and then centrifuged at 2000×g for 15 minutes at 4° C. to obtain plasma and then immediately stored at −20° C. until further analysis.

A few days before the end of each experiment, the animal body condition score (BCS), body length, wither's height, and heart girth were determined. All feed was then withdrawn from all animals in both treatments for 24 hours. They remained with access to water. After 24 hours, the weight was taken to determine fasting weight loss.

After 121 days in the first experiment and 135 days in the second, the animals were slaughtered. Biopsies were obtained from the left lobe of the liver and immediately put in liquid nitrogen and later stored at −80° C. Also, thin slices were taken from the liver and fixed in 4% formaldehyde (Bio-Lab Ltd., Jerusalem, Israel) for histological analysis. Around 150 g of the liver was put in a zip-lock bag and stored on dry ice and later in −20° C. for fat extraction and analysis. Liver-fat and glycogen content quantification, as well as histological analyses of hematoxylin-eosin (H&E) and periodic acid-Schiff (PAS) stained sections were examined blindly by an expert veterinary pathologist as described (Kalyesubula et al., Scientific Reports, 2020).

Percentage of Fat and Triglyceride Concentrations in Liver Tissue.

Total fat extraction was carried using a slightly modified Folch method (BLIGH and DYER, 1959; Folch et al., 1957). One gr of liver tissue was homogenized in a 20 ml (2:1) chloroform (Sigma-Aldrich, Milwaukee, Wis., USA)-methanol (Bio-Lab Ltd., Jerusalem, Israel) mixture. The homogenate was then sonicated using the VCX 750 sonicator (Sonic and Materials Inc, Newtown Conn.) for 5 min, 5 sec on 5 sec off, at an amplitude of 30. Sonicated samples were agitated for 24 h on an orbital shaker at room temperature, then centrifuged at 3000×g for 10 minutes. The top liquid phase was collected and washed with 0.9% NaCl solution, then centrifuged at 2500×g for 10 minutes. The resulting upper phase was discarded, and the remaining top interface was washed twice with the methanol-water mixture (1:1). The lower chloroform phase containing lipids was then evaporated under vacuum in a rotary evaporator. The fat was finally oven-dried to remove any remaining moisture at 45° C. for 2.5 hours. The fat weight was then determined, and the fat content was calculated as the percentage of the wet liver weight. Dried fat samples were stored in −20° C. until further analysis.

The liver triglycerides levels were also measured using a Triglyceride Quantification Kit (Abcam, Tel Aviv, Israel, ab65336) following the manufacturer's instructions.

Liver Glycogen Analysis.

Total liver glycogen was isolated using a modified method as described (Roe et al., 1961). Five grams of frozen liver sliced into small pieces were transferred into 5 ml of 10% Trichloroacetic acid (Sigma-Aldrich, Milwaukee, Wis., USA) and then homogenized (Tsiangtai machinery industry Co LTD, Taiwan). The homogenate was centrifuged, and the supernatant was transferred into a clean tube, centrifuged again to remove residual pellets. An equal volume of 95% ethanol was added to the supernatant above. The mixture was vortexed and incubated overnight at ˜25° C. for the glycogen to precipitate. It was then centrifuged, and the resulting supernatant was discarded. The glycogen pellet was further washed with 15 ml of 63% ethanol, followed by centrifugation and supernatant removal. The pellet was rewashed with 3 ml 95% ethanol. Finally, 3 ml of diethyl ether (Sigma-Aldrich, Milwaukee, Wis., USA) was added to the washed pellet, followed by centrifugation to remove the supernatant. All centrifugations were done at 3000×g for 5 min at 4° C. The glycogen pellet was finally oven-dried to remove any remaining moisture at 35° C. for 1 hour. The glycogen was weighted, and the glycogen liver content was calculated as the percentage of the wet liver weight.

Plasma Biochemical Analysis.

Plasma NEFA concentrations were analyzed using a NEFA kit (Wako Chemicals, GmbH, Neuss, Germany), and plasma insulin concentrations were analyzed by RIA (Diagnostic Products, Los Angeles, Calif.) according to the manufacturer's instructions. Plasma triglycerides were determined using the Cobas C111 analyzer (Roche Diagnostics, Rotkuez, Switzerland). Plasma thiamine concentrations were measured using an enzyme-linked immunosorbent assay (ELISA) method using vitamin B1 ELISA kit (Aviva Systems Biology, San Diego, Calif.); all following the manufacturer's instructions.

Determination of mRNA by Quantitative Polymerase Chain Reaction

To obtain RNA from leukocytes, blood was collected from sheep on day 126 via venipuncture into tubes with EDTA and kept on ice and then extracted RNA from the leukocytes using a Norgen leukocyte RNA purification kit (Norgen Biotek Corp., Ontario, Canada) according to the manufacturer's instructions. DNase treatment was carried out using Promega RQ1 RNase-Free DNase (Promega, Madison, Wis., USA). cDNA was synthesized from 500 ng total RNA using a Revert Aid RT-PCR Kit (Thermo Fisher Scientific, USA).

For the liver tissue, total RNA was isolated using a Norgen animal tissue RNA purification kit (Norgen Biotek Corp., Ontario, Canada) following the manufacturer's instructions. cDNA was synthesized from 1 μg total RNA using a Revert Aid RT-PCR Kit (Thermo Fisher Scientific, USA) and an Applied Biosystems 2720 Thermal Cycler (Thermo Fisher Scientific, USA) following the manufacturer's instructions. RT-qPCR analysis was carried out using 5×HOT FIREPol EvaGreen qPCR Supermix (Solis BioDyne, Tartu, Estonia). The reaction was composed of: 4 μl of cDNA, 0.3 ul of each primer designed using NCBI primer blast, 4 μl of 5×HOT FIREPol EvaGreen qPCR Supermix, completed with ultra-pure water (Biological Industries, Kibbutz Beit Ha'emmek, Israel) to a final volume of 20 μl, according to the manufacturer's instructions. RT-qPCR was carried out using a Rotor gene Q instrument (Qiagen, Hilden, Germany) under the following conditions: 95° C. for 12 minutes, 40 cycles of 95° C. for 15 seconds, 60 for 20 seconds and 72° C. for 20 seconds. Relative gene expression was computed using the ΔΔCT method 85 with the mean of the HC group as the normalizer. For leukocytes, the geometric mean of two reference genes (GAPDH and YWHAZ) was employed while for the liver, the geometric mean of three reference genes (YWHAZ, PPIA, and RPL19) was employed.

Statistical Analysis.

For experiment #1: Data of continuous dependent variables (body weight, liver weight, glucose, triglycerides, insulin, hepatic glycogen and hepatic fat) were analyzed with the repeated measures ANOVA using JMP (Version 14.0.0, SAS Institute Inc., Cary, N.C., 2016). The fixed factors of the model were: treatment, time and treatment X time, with a random factor (individual animal) nested within the treatment. Post-hoc pairwise comparisons at specific time points were done via contrast t-tests, accounting for multiple comparisons via Bonferroni correction. Differences between treatments in variables where time was not considered were determined by Student's t-tests or two-way ANOVA for the three treatments. Unless otherwise stated, data are summarized as means±standard error (SE), and a significance level of 0.05 was employed.

For experiment #2: Data of continuous dependent variables (glucose, insulin, triglycerides, NEFA and weight) were analyzed by repeated-measures ANOVA with the linear mixed model approach in JMP (Version 14.0.0, SAS Institute Inc., Cary, N.C., 2016). The model included Treatment (diet (LC vs. HC) and thiamine (THC vs. HC) as a between-subject fixed factor, Time (from treatment initiation) as a nominal within-subject fixed factor, Treatment by Time interaction, and Individual Animal as a random factor nested within Treatment. The distributions of model residuals were visually confirmed for normality. Post-hoc pairwise comparisons between treatments at specific time points were made by Student's t-test and Bonferroni-Holm corrected for multiple comparisons. Differences between treatments for other response variables lacking the time dimension, were determined by One-way ANOVA. Selected a priori comparisons to investigate the effects of diet (HC vs. LC) and thiamine treatment (HC vs. THC) were carried out using contrast t-tests. Two-tailed P-values are reported throughout. Data are presented as means±standard errors (SE), unless otherwise stated. A significance level of α=0.05 was employed.

Example 1. Sheep as a large animal model for hepatic steatosis. This has already been published (Kalyesubula et al., Scientific Reports, 2020).

HC Diet Induced Hyperglycemia.

Within a few days, the HC diet induced substantially higher blood glucose concentration than the LC diet (100 vs. 71 mg/dL; P<0.0001). Although the glycemic difference mildly decreased with time (FIG. 1), it remained relatively high and steady throughout the entire experiment with 4-months overall mean glucose concentrations of 86.2 vs. 68.6 mg/dL (P<0.0001), respectively. Noteworthy, blood glucose concentrations of ˜100 mg/dL are considered normal in humans; they are in fact extremely high in the context of sheep that have a normal blood glucose average of ˜52 mg/dL.

The HC Diet Induced Adiposity, Dyslipidemia and Hepatomegaly.

Lambs on the HC diet gained more weight than lambs on the LC diet (daily average of 392 vs. 165 gr; P<0.0001), reaching mean final body weights of 73 vs. 45 kg (P<0.0001), respectively. The respective final body condition scores of 3.7 vs. 2.5 indicate that the HC lambs developed significant adiposity (P<0.0001). Consistent with their enhanced adiposity, within 4 months, the HC diet induced increased plasma triglycerides concentration (FIG. 2) with mean final concentrations twice that of the LC lambs (31.8 vs. 15.6 mg/dL; P<0.0001). Such dyslipidemia in association with enhanced adiposity represents a common metabolic phenotype of obesity and MetS in humans.

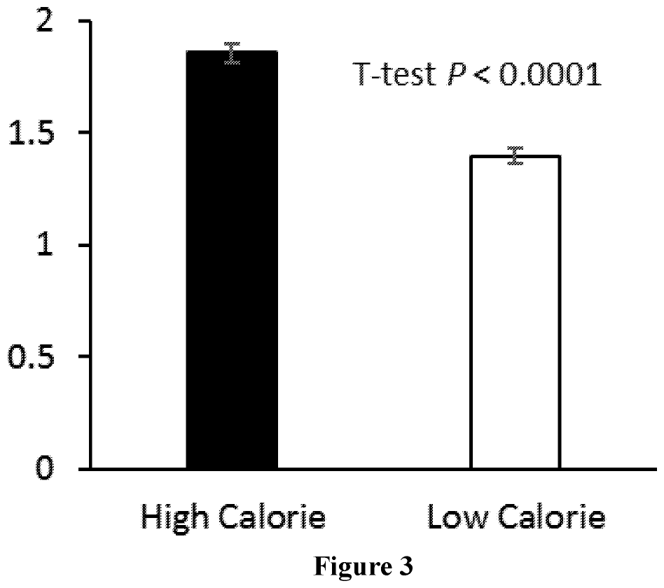

Surprisingly, lambs in the HC group developed not only larger livers (mean weight of 1.36 vs. 0.62 kg; P<0.0001), but also a greater hepatic index (liver weight normalized to body weight; FIG. 3) with values of 1.9 vs. 1.4 (P=0.0005), respectively, indicative of hepatomegaly.

The HC Diet Induced Insulin Resistance.

Hyperglycemic lambs grown on the HC diet had consistently higher concentrations of plasma insulin than on the LC diet; with a mean of 182.6 vs. 24.3 μIU/mL, P<0.0001 (FIG. 4). Interestingly, within 4-months of the dietary treatment, despite a similar fasting glucose levels for the HC and LC groups (FIG. 1), the HC group still had a significantly higher fasting insulin levels (49.1 vs. 20.5 μIU/mL, P=0.0002, FIG. 4). In other words, the HC group required higher insulin levels to maintain the same blood glucose levels, which implies that they are systemically more resistant to insulin. This is also indicated quantitatively by their fasting homeostatic model assessment of insulin resistance (HOMA-IR) values of 7.3 compared to 3.1 μIU*mg/[mL]² in the LC group (P=0.0008).

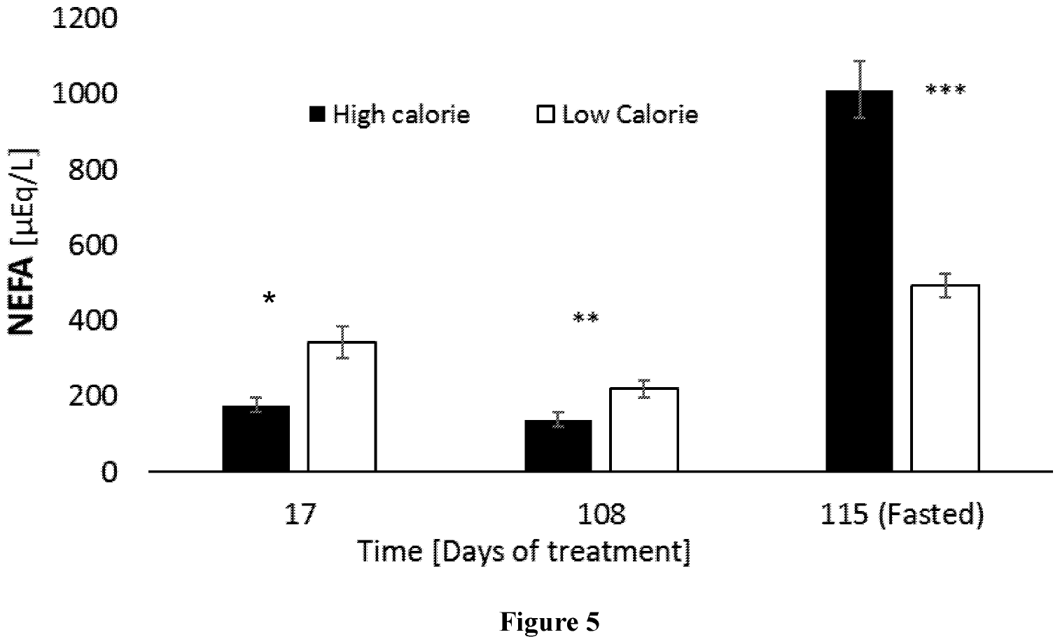

Moreover, since insulin negatively regulates adipose lipolysis, it is expected that higher insulin levels will exert lower plasma non-esterified fatty acids (NEFA). Intriguingly, despite the higher fasting insulin levels in the HC group, their plasma NEFA levels were significantly higher than the LC group (1010 vs. 492 μEq/L, ***P<0.0001, FIG. 5), this time indicating adipose tissue insulin resistance.

The HC Diet Induced Significant Hepatic Steatosis.

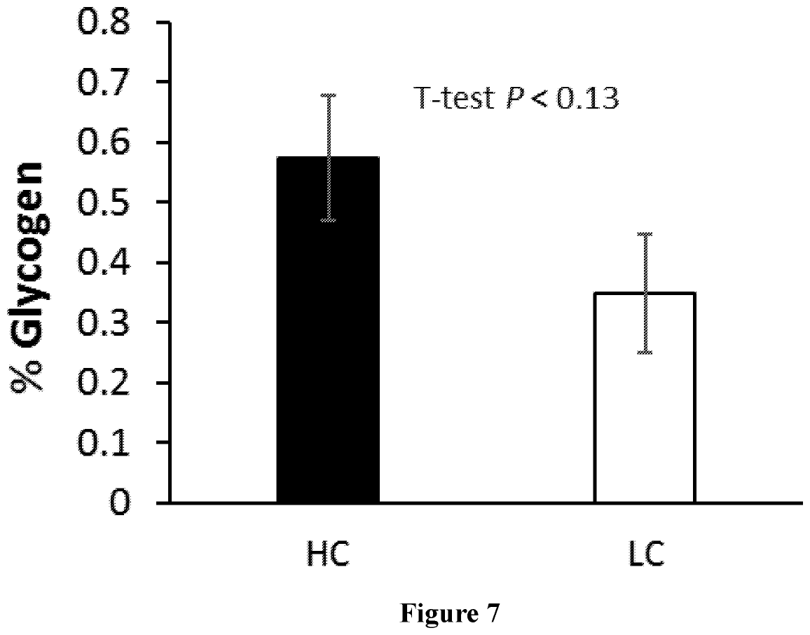

Strikingly, the HC group developed substantial hepatic steatosis with a liver fat content of 8.1 vs. 5.1% in the LC group (P<0.0001; FIG. 6A). By histopathology (FIG. 6B), the HC group had significantly greater macro-vesicular hepatocyte steatosis than the LC group (steatotic score of 2.1 vs. 0.4 P<0.0002). Occasional lobular inflammation, potentially mild ballooning was also observed among the HC lambs, but no significant fibrosis. The HC group tended to have greater hepatic glycogen content than the LC group (mean of 0.57 vs. 0.35%; FIG. 7), but the difference was not statistically different (P=0.1308).

Hepatic steatosis was strongly associated with the hepatic index (indicating an association between steatosis and hepatomegaly) as measured by the Spearman's correlation coefficient (r=0.64; p<0.0001). Among all the measured metabolic factors, the hepatic fat content was most strongly associated with the mean blood glucose levels (r=0.755; P<0.0001). The next strongly associated factor was mean insulin (r=0.714; P<0.0001), while the correlation with insulin resistance (fasting HOMA-IR), which is often considered as the most significant predictor of NAFLD was slightly lower (r=0.683; P<0.0001). Hepatic steatosis was not associated with fasting plasma-concentrations of glucose (r=0.098; P=0.61) nor of triglycerides (r=0.198; P=0.146). Surprisingly, hepatic steatosis was negatively correlated with plasma NEFA (r=−0.5753, P=0.0009). This may indicate that hepatic de novo lipogenesis, rather than circulating NEFA released from adipose tissue, may have served as the main source for fat accumulated in the liver.

### Example 2. Effects of Thiamine Treatment on Hepatic Steatosis

To test the potential of thiamine to reduce hepatic steatosis, 36 male weaned lambs were randomly assigned to three treatment groups. One group (LC; n=12) was raised on the LC diet to represent sheep with lean livers. Two additional groups (n=12 ea.) were raised on the HC diet for two months to an average weight of 46.5 Kg before the thiamine treatment was initiated for 2.5 months in combination with the HC diet. The thiamine-treated HC group (THC; n=12) was subcutaneously injected with an average of 18 mg of thiamine per Kg body weight per week, which is roughly equivalent to a daily dose of 150 mg/animal. Injections were initially given 3 times a week and then 5 times a week. The second HC group (n=12) was injected at the same frequency but with equal volumes of saline solution. To evenly account for potential injections stress effects, the LC group was also injected similarly with saline for the same duration.

Thiamine Treatment Decreased Blood Glucose Levels and Weight Gain.

Within one week of treatment, lower blood glucose levels (71.7 vs. 77.2 mg/dL) were measured in the thiamine treated HC group (THC; n=12) compared to the saline-treated HC group (HC; n=12). This difference in the glycemic index was maintained for 3 weeks, and then gradually decreased as the lambs kept on gaining more bodyweight and almost diminished after six weeks (FIG. 8). Increasing the frequency of treatment from 3 to 5 times a week, re-established the glycemic index difference between the HC and THC groups.

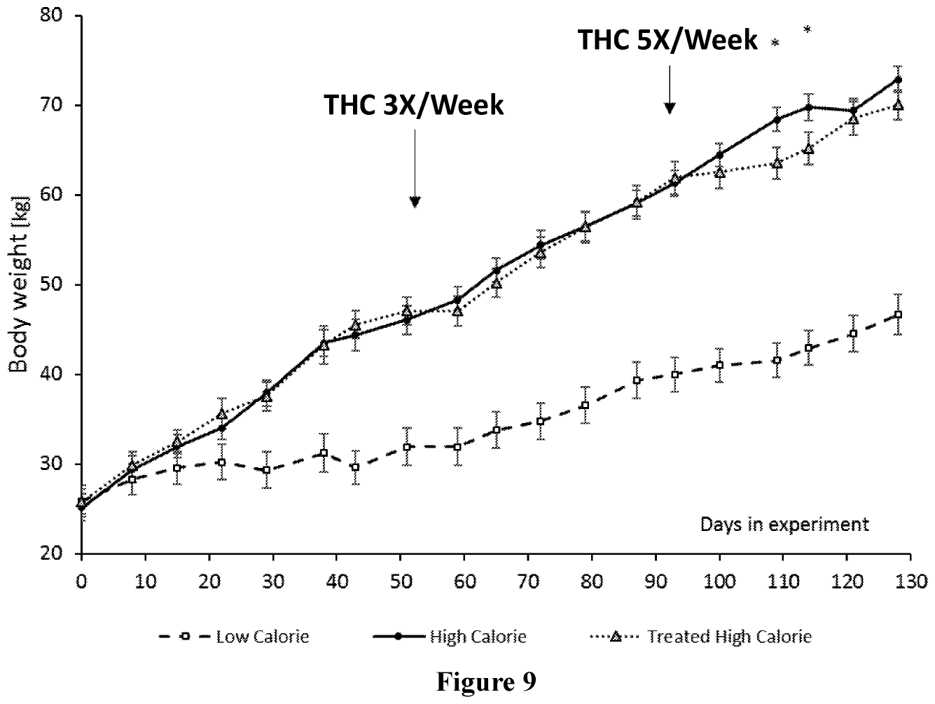

Since blood glucose values are related to growth rates in lambs, a similar trend was observed for the effect of thiamine on weight gain (FIG. 9). Here a smaller difference was observed with 3×/week treatment but more significant in response to the 5×/week frequency of treatment. Importantly, these effects on blood glucose and weight gain were measured, although the average feed intake was not different between the HC and THC groups (FIG. 10).

Thiamine Treatment Prevented Hepatic Steatosis and Increased Glycogen Content.

As can be seen in FIG. 11A the thiamine treatment dramatically decreased the hepatic fat content to levels below the steatosis threshold, which were statistically indistinguishable from those in the lean livers of the LC lambs. Hepatocellular steatosis, either as macro-vesicular or micro-vesicular, which was correlated with advanced histology of NAFLD, was significantly increased in the HC compared to both the LC and THC groups (FIG. 11B, FIG. 11D). As observed before (Kalyesubula et al., Scientific Reports 2020), the HC diet increased the hepatic index (liver weight/BW), yet thiamine had no significant effect on it (FIG. 11C). Therefore, since the THC animals presented with lean livers, the observed diet-induced hepatomegaly seen in both the HC and THC animals was not correlated with the hepatic fat-content, and potentially more related to the overall organ growth in cell count and mass.

Markedly, despite the reducing effect of thiamine on blood glucose (FIG. 1), the hepatic glycogen levels in the THC group increased compared to both the LC and HC groups (FIG. 12). This may indicate a shift from lipogenesis to gluconeogenesis, yielding a desirable shift in hepatic-energy storage from the fat form to glycogen form.

The High-calorie diet decreased the liver expression of genes involved in mitochondrial catabolism.

Mammalian regulation of energy expenditure is primarily mediated by sensing the cellular levels of adenosine monophosphate (AMP) and NAD⁺, respectively, by AMP-activated protein kinase (AMPK) and sirtuin 1 (SIRT1). In the setting of low-caloric intake, the resulting increased levels of AMP and NAD⁺ allosterically activate AMPK and SIRT1, respectively. The activation of these evolutionary-conserved sensors turns signaling cascades for stimulation of catabolic and inhibition of anabolic processes. Peroxisome proliferator-activated receptor coactivator-1 alpha (PGC-1α), a master regulator of mitochondrial biogenesis, is directly targeted by SIRT1 and AMPK to stimulate mitochondrial oxidative processes in response to energy needs and nutrients availability.

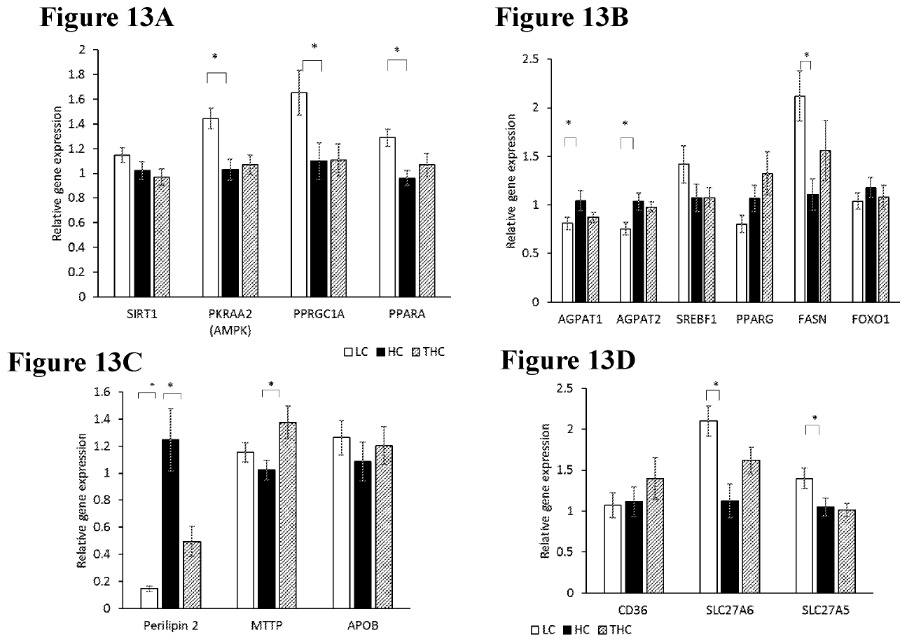

In the current study, SIRT1 expression was not different between the treatments. However, the overnourished sheep raised on the HC diet exhibited decreased expression of the genes coding for AMPK (PRKAA2), PGC-1α (PPARGC1A), and for peroxisome proliferator-activated receptor alpha (PPAR-α)-PPARA (P=0.05; FIG. 13A). Reduced expression of this molecular network is expected to promote DNL and to inhibit FA oxidation, which is consistent with the increased hepatic steatosis observed in the HC animals here and previously. Thiamine did not affect the expression levels of these genes (FIG. 13B).

Hepatic Gene Expression Alterations by Thiamine Favored Inhibition of Fat Storage.

Under energy abundance conditions, as induced by the HC diet, newly derived TG from hepatic synthesis and from liver uptake of circulating lipoproteins can be either secreted to the bloodstream in very-low-density lipoprotein (VLDL) particles, or incorporated to cytosolic lipid droplets (LD) for storage as intrahepatic fat. Expansion or reduction of the fat content in LD is dynamically regulated by LD-associated proteins and by the availability of cytosolic TG, which is partly controlled by the lipidation of VLDL particles by microsomal triglyceride transfer protein (MTP) encoded by the MTTP gene. Hepatic steatosis has been associated with genetic defects in both MTP and ApoB100, the hepatic VLDL lipoprotein. In the current study, thiamine increased the abundance of the MTTP transcripts compared with the untreated HC group (P=0.001; FIG. 3C). No effects were detected on APOB (FIG. 13C).

Perilipins are the predominant hepatocellular LD proteins. These surface-associated LD proteins stabilize the LD structure and control substrate availability for certain LD-associated enzymes⁴³. Perilipin 2, which positively correlates with hepatic steatosis, is one of the most characterized LD proteins in fatty liver disease. Here, the HC treatment significantly increased the abundance of Perilipin 2 transcripts compared to the LC treatment (P<0.0001; FIG. 13C). Remarkably, the thiamine treatment lowered Perilipin 2 transcripts substantially (P=0.002; FIG. 13C). These findings are in accordance with studies showing that both mRNA and protein levels of Perilipin 2 increased with hepatic TG accumulation, while gene inactivation of perilipin 2 lowered hepatic steatosis.

The low-calorie diet increased the expression of genes involved in liver uptake of fatty acids.

A variety of proteins associated with hepatic steatosis were implicated in the liver uptake of circulating NEFA, including FA translocase/CD36, caveolin, and FA transport protein (FATP) complexes, which possesses very-long-chain acyl-CoA synthase activity. Consistent with the elevated plasma NEFA concentrations in the LC animals (FIG. 1C), their mRNA abundance of FATP6 (SLC27A6) (P=0.0008), FATP5 (SLC27A5) (P=0.03) was higher than in the HC animals (FIG. 3D). As PPARα positively regulates the transcription of NEFA transporters, these data are consistent with the observed higher expression of PPARα (FIG. 13D). No effect of thiamine was observed on the expression of this energy-sensing axis or on the transporters of NEFA.

The low hepatic-fat content in the LC lambs (FIG. 2), therefore, suggests that the NEFA influx was utilized primarily for energy production, which is consistent with their increased expression of genes promoting FA oxidation (FIG. 13A).

Hepatic fat accumulation was associated with altered gene expression of proinflammatory cytokines and antioxidants; some were reversed by thiamine.

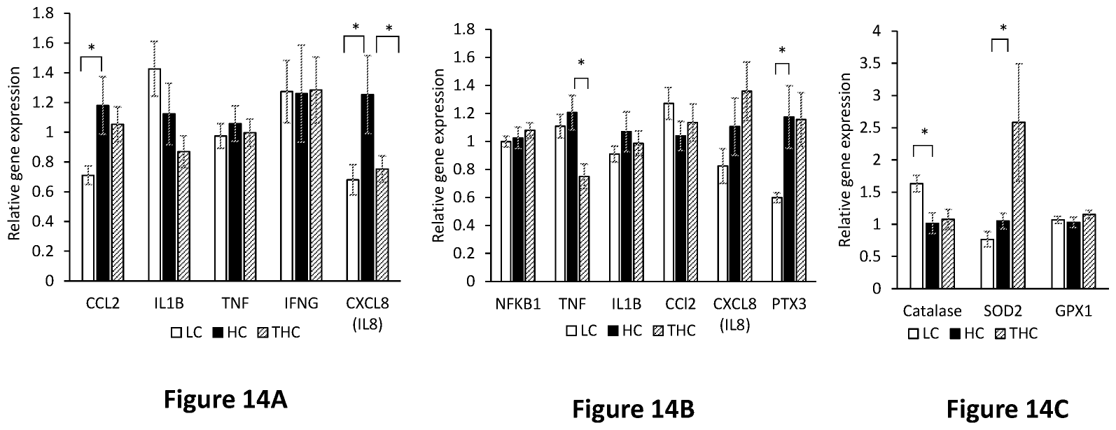

As a well-documented driver of the pathogenesis of obesity, MetS, and insulin resistance, inflammation also plays a role in the progression of MAFLD. In addition to the common histology signs of steatohepatitis, inflammation in MAFLD is manifested by increased circulating proinflammatory cytokines and leukocytes that can infiltrate the liver to fuel local and systemic inflammatory processes. In our study, sheep fed the HC diet exhibited increased mRNA abundance of CCL2 and CXCL8 (P=0.05; FIG. 14A) in circulating leukocytes compared with the LC-fed animals. Conversely, thiamine-treated animals had lower expression of CXCL8 (P=0.05; FIG. 14A). No treatment effects were detected for IL1B, TNF, and IFNG.

In the liver, the HC diet increased the mRNA levels of PTX3 compared with the LC diet (P=0.01; FIG. 14B). The thiamine treatment lowered the expression of TNF (P=0.005; FIG. 14B). Interestingly, mice treated with thalidomide, an anti-TNF-α drug, exhibited improvements in hepatic alterations caused by a high-fat diet. No significant alterations were detected in the gene expression of NFKB1, IL1B, CCL2, and IL8 (FIG. 14B).

Overnutrition and hepatic steatosis are associated with an increase in reactive oxygen species and oxidative stress, which may lower endogenous enzymatic antioxidants. Consistently, the expression of catalase was lower as a result of the HC diet (P=0.005; FIG. 14c). Whereas the thiamine treatment did not affect the expression of catalase, it did increase the expression levels of superoxide dismutase 2 (SOD2) (P=0.02) (FIG. 14C). There were no detected treatment effects on the expression of glutathione peroxidase 1 (GPX1).

While the invention has been described with reference to various embodiments, it should be understood by those skilled in the art that various changes may be made, and equivalents may be substituted for elements thereof without departing from the essential scope of the invention. In addition, many modifications may be made to adapt a situation or material to the teachings of the invention without departing from the essential scope thereof. Therefore, it is intended that the invention is not limited to any embodiment disclosed herein contemplated for carrying out this invention, but that the invention will include all embodiments falling within the scope of the claims.

1. A method of preventing or treating a condition selected from the group consisting of:
(i) fatty liver (FL) disease (FLD),
(ii) a FL-associated disease, and
(iii) a FL-associated symptom,
in a patient in need of such prevention or treatment, the method comprising the step of administering a therapeutically effective amount of thiamine, a thiamine ester, or a thiamine analog, to the patient.

2. (canceled)

3. The method of claim 1, wherein the condition is a FL-associated disease selected from the group consisting of non-alcoholic fatty liver disease (NAFLD), alcoholic fatty liver disease, non-alcoholic steatohepatitis (NASH), metabolic syndrome (MetS), type-2 diabetes mellitus (T2D), cardiovascular disease, dyslipidemia, alcoholic liver disease, hepatocellular carcinoma (HCC), viral steatohepatitis, overnutrition, overweight, and obesity.

4. The method of claim 1, wherein the condition is a FL-associated symptom selected from the group consisting of hepatic steatosis, hepatic cirrhosis, hepatic inflammation, hepatic fibrosis, hepatic pain, hepatic yellow pigmentation, decreased liver function, low liver glycogen level, tiredness, systemic insulin resistance, hyperglycemia, systemic hypertriglyceridemia, micro-vesicular steatosis, macro-vesicular steatosis, high food intake, and high weight gain.

5. The method of claim 4, wherein the hepatic steatosis is at least 5.5% by weight fat content (wet/wet).

6. The method of claim 1, wherein the patient is afflicted with Type-2 diabetes mellitus (T2D) and/or insulin resistance.

7. The method of claim 1, wherein the patient is not afflicted with Type-2 diabetes mellitus (T2D) and/or insulin resistance.

8. The method of claim 1, comprising administering between about 200 mg to about 500 mg thiamine, a thiamine ester, or a thiamine analog, every day.

9. The method of claim 1, wherein the thiamine, a thiamine ester, or a thiamine analog, is administered to the patient by oral administration, intravenous administration or intramuscular administration.

10-12. (canceled)

13. The method of claim 1, wherein the thiamine ester is selected from the group consisting of thiamine monophosphate and thiamine pyrophosphate.

14. (canceled)

15. The method of claim 1, wherein the thiamine analog is selected from the group consisting of Acefurtiamine, Acetiamine, Allithiamine, Beclotiamine, Benfotiamine, Bentiamine, Bisbentiamine, Cetotiamine, Cycotiamine, Fursultiamine, Monophosphothiamine, Octotiamine, Prosultiamine, Sulbutiamine, and Vintiamol.

16. A composition comprising thiamine, a thiamine ester, or a thiamine analog, and at least one additional agent selected from the group consisting of an insulin sensitizer, an insulin-releasing enhancer, and an antioxidant.

17. The composition of claim 16, wherein the insulin sensitizer is selected from the group consisting of a Biguanide, a Thiazolidinedione, and a Lyn kinase activator; or the insulin-releasing enhancer is a glucagon-like peptide-1 receptor (GLP-1 receptor) agonist, or the antioxidant is vitamin E, or any combination thereof.

18. The composition of claim 17, wherein the Biguanide is Metformin, or the Thiazolidinedione is Pioglitazone, or the Lyn kinase activator is Tolimidone, or any combination thereof.

19-21. (canceled)

22. The composition of claim 17, wherein the GLP-1 receptor agonist is liraglutide.

23. (canceled)

24. The composition of claim 16, comprising between about 200 mg to about 500 mg thiamine, a thiamine ester, or a thiamine analog.

25-26. (canceled)

27. The composition of claim 26, wherein the thiamine ester is selected from the group consisting of thiamine monophosphate and thiamine pyrophosphate.

28. (canceled)

29. The composition of claim 26, wherein the thiamine analog is selected from the group consisting of Acefurtiamine, Acetiamine, Allithiamine, Beclotiamine, Benfotiamine, Bentiamine, Bisbentiamine, Cetotiamine, Cycotiamine, Fursultiamine, Monophosphothiamine, Octotiamine, Prosultiamine, Sulbutiamine, and Vintiamol.

30. (canceled)

31. A method for inducing fatty liver (FL), a FL-associated disease, or a FL-associated symptom, in a large animal, comprising the step of administering a high-calorie diet to the animal.

32. The method of claim 31, wherein the animal is a sheep, and wherein the high-calorie diet comprises an average daily metabolizable energy of 6.3 MCal.

33. (canceled)

34. The method of claim 31, further comprising administering a drug to the animal, and testing the effect of the drug on the fatty liver (FL), the FL-associated disease, or the FL-associated symptom, in the animal.

35. (canceled)
